(19) United States
(12) Patent Application Publication
(10) Pub. No.: US 2005/0130174 A1
(43) Pub. Date: Jun. 16, 2005

(54) Label-free gene expression profiling with universal nanoparticle probes in microarray assay format

(75) Inventors: Yijia P. Bao, Mount Prospect, IL (US); Uwe R. Muller, Waukegan, IL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Nanosphere, Inc., Northbrook, IL (US)

(21) Appl. No.: 10/789,831

(22) Filed: Feb. 27, 2004

Related U.S. Application Data
(60) Provisional application No. 60/450,268, filed on Feb. 27, 2003.

(51) Int. Cl. C12Q 1/68; C12M 1/34

(52) U.S. Cl. 435/6; 435/287.2

(57) ABSTRACT

A method and kit for the label-free detection of global gene expression using nanoparticle probes in an array assay format is described. The method avoids the problems associated with fluorescent labeling and target amplification.

### CROSS-REFERENCE

This application claims the benefit of priority from U.S. Provisional application No. 60/450,268, filed Feb. 27, 2003, which is incorporated by reference in its entirety.

### FIELD OF THE INVENTION

The present invention relates to a method and kit for label-free detection of global gene expression using a universal nanoparticle probe in a microarray assay format.

### BACKGROUND OF THE INVENTION

Currently, the mRNA target samples are typically labeled directly with fluorescent dyes (e.g., Cy3 or Cy5) or indirectly with other molecules (e.g., biotin) for expression analysis. Because of the limitation in sensitivity with fluorescence based detection, mRNA targets are often amplified and then labeled before hybridization to microarrays. For example, mRNA targets are converted to double-stranded cDNA with RNA reverse transcriptase and DNA polymerase. Then T7 RNA polymerase is used for in vitro transcription to amplify targets. Furthermore, the RNA targets are labeled either during the in vitro transcription or thereafter. This RNA labeling and amplification process is laborious, costly and time consuming. Moreover, the rate of amplification may differ for different genes, the labeling efficiency of different dye molecules may vary, and the hybridization kinetics of cDNAs containing different dyes may differ, all of which may result distort the accuracy of expression analysis.

To date, there is a need for a method for detecting gene expression that avoids the problems associated with fluorescent labeling and amplification. The proposed label-free expression analysis with universal nanoparticle probes eliminates the RNA labeling process. The higher sensitivity of the nanoparticle probes also reduces the need for target amplification.

### SUMMARY OF THE INVENTION

The present invention relates to a method and kit for label-free detection of global gene expression using a nanoparticle probe in a array assay format. The gold-nanoparticle probes with silver enhancement have been found to allow for high specificity and sensitivity detection.

In one embodiment of the invention, a method is provided for detecting or quantitating gene expression in a sample, said sample believed to have one or more different types of unlabeled target nucleic acids, each type of target nucleic acid having an oligonucleotide tail, said method comprising: providing a substrate having a plurality of types of capture nucleic acid sequences attached thereto in an array for the detection of multiple portions of a target nucleic acid, the detection of multiple different target nucleic acids, or both; providing nanoparticles having oligonucleotides bound thereto, the oligonucleotides bound to the nanoparticles having a sequence that is complementary to at least a portion of the oligonucleotide tail; contacting the sample, the substrate, and the nanoparticles, said contracting occurring under conditions effective for hybridization of the target nucleic acids to the capture nucleic acid sequences bound to the substrate and hybridization of the target nucleic acids to the nanoparticles; and observing a detectable change.

In one aspect of the invention, the target nucleic acid may be RNA, e.g., mRNA, or DNA, e.g., cDNA. The oligonucleotide tail comprises a poly dT, a poly dA, or a synthetic oligonucleotide having a predetermined sequence. The oligonucleotides bound to the nanoparticles comprises a poly dT, a poly dA, or a synthetic oligonucleotide having a predetermined sequence. The capture nucleic acid sequences comprise an oligonucleotide, cDNA, or genomic sequence fragment. The nanoparticles may be made of gold.

In another aspect of the invention, the sample is first contacted with the substrate, said contacting occurring under conditions effective for hybridization of the target nucleic acids with the capture nucleic acid sequence bound to the substrate, and then contacting the target nucleic acid bound to the substrate with the nanoparticles, said contacting occurring under conditions effective for hybridization of the target nucleic acids bound to the substrate with the oligonucleotides bound to the nanoparticles.

In another aspect of the invention, the sample is first contacted with the nanoparticles, said contacting occurring under conditions effective for hybridization of the target nucleic acids with the oligonucleotides bound to the nanoparticles, and then contacting the target nucleic acid bound to the nanoparticles with the substrate, said contacting occurring under conditions effective for hybridization of the target nucleic acids bound to the nanoparticles with the capture nucleic acid sequences bound to the substrate.

In another aspect of the invention, the sample, nanoparticles and substrate are contacted simultaneously under conditions effective for hybridization of the target nucleic acids with the oligonucleotides bound to the nanoparticles and with the capture nucleic acid sequences bound to the substrate.

In another aspect of the invention, the detectable change is observed after contacting the substrate having target nucleic acids and nanoparticles with a staining material. The staining material may be silver stain or any suitable staining material.

In another aspect of the invention, a method is provided for detecting or quantitating gene expression in a sample, said sample believed to have one or more different types of unlabeled target ribonucleic acids, each type of target ribonucleic acid including a poly dA olignonucleotide tail or a synthetic oligonucleotide tail of a predetermined sequence, said method comprising: providing a substrate having a plurality of types of capture nucleic acid sequences attached thereto in an array for the detection of multiple portions of a target ribonucleic acid, the detection of multiple different target ribonucleic acids, or both; providing nanoparticles having bound thereto poly dT oligonucleotides or a synthetic oligonucleotide sequence complementary to the sequence of the oligonucleotide tail; contacting the sample, the substrate, and the nanoparticles, said contracting occurring under conditions effective for hybridization of the target ribonucleic acids to the capture nucleic acid sequences to the substrate and hybridization of the target ribonucleic acids to the nanoparticles; and contacting the nanoparticles bound to the support with a staining material to produce a detectable change; and observing the detectable change.

In one aspect of the invention, the sample is first contacted with the substrate, said contacting occurring under conditions effective for hybridization of the target ribonucleic acids with the capture nucleic acid sequences bound to the substrate, and then contacting the target ribonucleic acid bound to the substrate with the nanoparticles, said contacting occurring under conditions effective for hybridization of the target ribonucleic acids bound to the substrate with the oligonucleotides bound to the nanoparticles.

In another aspect of the invention, the sample is first contacted with the nanoparticles, said contacting occurring under conditions effective for hybridization of the target ribonucleic acids with the oligonucleotides bound to the nanoparticles, and then contacting the target ribonucleic acid bound to the nanoparticles with the substrate, said contacting occurring under conditions effective for hybridization of the target ribonucleic acids bound to the nanoparticles with the capture nucleic acid sequences bound to the substrate.

In another aspect of the invention, the sample, nanoparticles and substrate are contacted simultaneously under conditions effective for hybridization of the target nucleic acids with the oligonucleotides bound to the nanoparticles and with the capture nucleic acid sequences bound to the substrate.

In another aspect of the invention, the nanoparticles are made of gold.

In another aspect of the invention, the staining material is silver stain or any suitable material.

In another aspect of the invention, the capture nucleic acid sequences comprise an oligonucleotide, cDNA, or genomic sequence fragment.

In another embodiment of the invention, a method is provided for detecting or quantitating gene expression in a sample, said sample believed to have one or more different types of target cDNAs, each type of target cDNA including a poly dT olignonucleotide tail or a synthetic oligonucleotide tail having a predetermined sequence, said method comprising: providing a substrate having a plurality of types of capture nucleic acid sequences attached thereto in an array for the detection of multiple portions of a target ribonucleic acid, the detection of multiple different target ribonucleic acids, or both; providing nanoparticles having bound thereto poly dA oligonucleotides or synthetic oligonucleotides having a predetermined sequence; contacting the sample, the substrate, and the nanoparticles, said contracting occurring under conditions effective for hybridization of the target cDNAs to the capture nucleic acid sequences bound to the substrate and hybridization of the target cDNAs to the nanoparticles; and contacting the nanoparticles bound to the support with a staining material to produce a detectable change; and observing the detectable change.

In one aspect of the invention, the sample is first contacted with the substrate, said contacting occurring under conditions effective for hybridization of the target cDNAs with the capture nucleic acid sequences bound to the substrate, and then contacting the target cDNAs bound to the substrate with the nanoparticles, said contacting occurring under conditions effective for hybridization of the target cDNAs bound to the substrate with the oligonucleotides bound to the nanoparticles.

In another aspect of the invention, the sample is first contacted with the nanoparticles, said contacting occurring under conditions effective for hybridization of the target cDNAs with the oligonucleotides bound to the nanoparticles, and then contacting the target cDNAs bound to the nanoparticles with the substrate, said contacting occurring under conditions effective for hybridization of the target cDNAs bound to the nanoparticles with the capture nucleic acid sequences bound to the substrate.

In another aspect of the invention, the target cDNAs, nanoparticles and substrate are contacted simultaneously under conditions effective for hybridization of the target cDNAs with the oligonucleotides bound to the nanoparticles and with the capture nucleic acid sequences bound to the substrate.

In another aspect of the invention, the nanoparticles are made of gold.

In another aspect of the invention, the staining material is silver stain or any other suitable material.

In another aspect of the invention, the capture nucleic acid sequences comprise an oligonucleotide, cDNA, or genomic sequence fragment.

In another embodiment of the invention, a kit is provided for detecting or quantitating gene expression in a sample., said sample believed to have one or more different types of unlabeled target nucleic acids, each type of target nucleic acid including a poly dT, poly dA oligonucleotide tail, or a synthetic oligonucleotide tail having a predetermined sequence, said kit comprising: a substrate having a plurality of types of capture nucleic acid sequences attached thereto in an array for the detection of multiple portions of a target nucleic acid, the detection of multiple different target nucleic acids, or both; and one or more types of nanoparticles having bound thereto poly dT oligonucleotides, poly dA oligonucleotides, or synthetic oligonucleotides having a predetermined sequence.

In one aspect of the invention, the nanoparticles are made of gold.

In another aspect of the invention, the capture nucleic acid sequences comprise an oligonucleotide, cDNA, or genomic sequence fragment.

These and other embodiments of the present invention will become evident from the following more detailed description of the invention and the claims.

### BRIEF DESCRIPTION OF THE FIGURES

FIG. 1 illustrates detection of fluorescent Cy3-labeled RNA targets in the (part A) presence or absence of gold universal nanoparticles functionalized with poly dA (20mer) using a test array substrate under one-step hybridization conditions; and (part B) the Cy3 signal was found to be similar for both conditions, supporting that there is no negative interference by nanoparticle probes on target hybridization.

FIG. 2 illustrates (part A) detection of fluorescent Cy3-labeled RNA targets by measuring Cys3 signals and nanoparticle scatter signals for each spot on the array after hybridization with the targets and universal nanoparticle probes under one-step or two-step hybridization conditions; and (part B) the Cys signal was found to be greater in the presence of nanoparticle probe than in the absence of the probe, regardless of whether a one- or two-step hybridization format was used.

FIG. 3 illustrates (part A) linear correlation between observed nanoparticle scatter signals and target concentration. No detectable scatter signal was observed with control (no target added). In (part B), a linear plot for nanoparticle signal scatter was observed, demonstrating a linear correlation between target concentration and nanoparticle concentration and the feasibility of using nanoparticles for detecting gene expression.

FIG. 4 illustrates (part A) a linear correlation between nanoparticle scatter singles and Cy3 fluorescent signal for target concentration ranges exceeding 3 logs; and (part B) signal to background noise ratio for the scatter signal was 10-40 fold higher than that for the Cy3 fluorescent signal at all target concentrations, demonstrating the higher sensitivity of nanoparticle labels in the assay system.

FIG. 5 illustrates the feasibility of applying universal nanoparticle probes for label-free human gene expression analysis using (A) capture human gene specific oligomers microarrayed at different concentrations and using different concentrations of formamide in the hybridization mixture. A reduction in probe-capture binding interference was observed with decreased concentrations of capture oligomers. Also, a reduction of non-specific signals was observed with increasing amounts of formamide.

FIG. 6 illustrates the results of two-step hybridizations on a test array which has been arrayed at different capture oligomer concentrations (1 uM, 3 uM and 9 uM). Total human RNA or control (no RNA) samples were then hybridized to the array. No probe-capture interaction was observed for all three capture oligomer concentrations. Also, no non-specific binding was observed (part A), even at low formamide concentrations in the hybridization buffer. In addition, human total RNA hybridization showed that signal intensities were comparable for all three capture oligomer concentrations (part B). Part C illustrates the layout for arrays shown in Parts A and B and in FIG. 7.

FIG. 7 illustrates the effect of different SDS detergent concentrations on hybridization. SDS concentrations were titrated from 0.001% to 0.1% and it was observed that scatter signal (part A) and hybridization signal (part B) increased as the SDS concentration increased from 0.001% to 0.2%.).

FIG. 8 illustrates a comparison of sensitivity of fluorescent based and gold nanoparticle based detection of a human beta actin gene with human gene test arrays after two hour hybridization (part A) or after overnight hybridization (part B). The results indicate that nanoparticle-based detection had a sensitivity that was 25-100 fold higher than fluorescent detection in a human RNA detection assay.

FIG. 9 illustrates the sensitivity of RNA detection and determination of a correlation between signal intensity and mRNA copy numbers after over-night hybridization or two-hour hybridization.

### DETAILED DESCRIPTION OF THE INVENTION

Unless otherwise required by context, singular terms shall include pluralities and plural terms shall include the singular.

As utilized in accordance with the present disclosure, the following terms, unless otherwise indicated, shall be understood to have the following meanings:

As used herein, a “nucleic acid sequence,” a “nucleic acid molecule,” or “nucleic acids” refers to one or more oligonucleotides or polynucleotides as defined herein. As used herein, a “target nucleic acid molecule” or “target nucleic acid sequence” refers to an oligonucleotide or polynucleotide comprising a sequence that a user of a method of the invention desires to detect in a sample.

The term “polynucleotide” as referred to herein means single-stranded or double-stranded nucleic acid polymers of at least 10 bases in length. In certain embodiments, the nucleotides comprising the polynucleotide can be ribonucleotides or deoxyribonucleotides or a modified form of either type of nucleotide. Said modifications include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

The term “oligonucleotide” referred to herein includes naturally occurring, and modified nucleotides linked together by naturally occurring, and/or non-naturally occurring oligonucleotide linkages. Oligonucleotides are a polynucleotide subset comprising members that are generally single-stranded and have a length of 200 bases or fewer. In certain embodiments, oligonucleotides are 10 to 60 bases in length. In certain embodiments, oligonucleotides are 12, 13, 14, 15, 16, 17, 18, 19, or 20 to 40 bases in length. Oligonucleotides may be single stranded or double stranded, e.g. for use in the construction of a gene mutant. Oligonucleotides of the invention may be sense or antisense oligonucleotides with reference to a protein-coding sequence.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotide linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al., 1986, Nucl. Acids Res., 14:9081; Stec et al., 1984, J. Am. Chem. Soc., 106:6077; Stein et al., 1988, Nucl. Acids Res., 16:3209; Zon et al., 1991, Anti-Cancer Drug Design, 6:539; Zon et al., 1991, OLIGONUCLEOTIDES AND ANALOGUES: A PRACTICAL APPROACH, pp. 87-108 (F. Eckstein, Ed.), Oxford University Press, Oxford England; Stec et al., U.S. Pat. No. 5,151,510; Uhlmann and Peyman, 1990, Chemical Reviews, 90:543, the disclosures of which are hereby incorporated by reference for any purpose. An oligonucleotide can include a detectable label to enable detection of the oligonucleotide or hybridization thereof.

An “addressable substrate” or “substrate” used in a method of the invention can be any surface capable of having oligonucleotides bound thereto. Such surfaces include, but are not limited to, glass, metal, plastic, or materials coated with a functional group designed for binding of oligonucleotides. The coating may be thicker than a monomolecular layer; in fact, the coating could involve porous materials of sufficient thickness to generate a porous 3-dimensional structure into which the oligonucleotides can diffuse and bind to the internal surfaces.

The term “capture oligonucleotide” as used herein refers to an oligonucleotide that is bound to a substrate and comprises a nucleic acid sequence that can locate (i.e. hybridize in a sample) a complementary nucleotide sequence or gene on a target nucleic acid molecule, thereby causing the target nucleic acid molecule to be attached to the substrate via the capture oligonucleotide upon hybridization. Suitable, but non-limiting examples of a capture oligonucleotide include DNA, RNA, PNA, LNA, or a combination thereof. The capture oligonucleotide may include natural sequences or synthetic sequences, with or without modified nucleotides.

A “detection probe” of the invention can be any carrier to which one or more detection oligonucleotides can be attached, wherein the one or more detection oligonucleotides comprise nucleotide sequences complementary to a particular nucleic acid sequence. The carrier itself may serve as a label, or may contain or be modified with a detectable label, or the detection oligonucleotides may carry such labels. Carriers that are suitable for the methods of the invention include, but are not limited to, nanoparticles, quantum dots, dendrimers, semi-conductors, beads, up- or down-converting phosphors, large proteins, lipids, carbohydrates, or any suitable inorganic or organic molecule of sufficient size, or a combination thereof.

As used herein, a “detector oligonucleotide” or “detection oligonucleotide” is an oligonucleotide as defined herein that comprises a nucleic acid sequence that can be used to locate (i.e. hybridize in a sample) a complementary nucleotide sequence or gene on a target nucleic acid molecule. Suitable, but non-limiting examples of a detection oligonucleotide include DNA, RNA, PNA, LNA, or a combination thereof. The detection oligonucleotide may include natural sequences or synthetic sequences, with or without modified nucleotides.

As used herein, the terms “label” refers to a detectable marker that may be detected by photonic, electronic, opto-electronic, magnetic, gravity, acoustic, enzymatic, or other physical or chemical means. The term “labeled” refers to incorporation of such a detectable marker, e.g., by incorporation of a radiolabeled nucleotide or attachment to an oligonucleotide of a detectable marker.

A “sample” as used herein refers to any quantity of a substance that comprises nucleic acids and that can be used in a method of the invention. For example, the sample can be a biological sample or can be extracted from a biological sample derived from humans, animals, plants, fungi, yeast, bacteria, viruses, tissue cultures or viral cultures, or a combination of the above. They may contain or be extracted from solid tissues (e.g. bone marrow, lymph nodes, brain, skin), body fluids (e.g. serum, blood, urine, sputum, seminal or lymph fluids), skeletal tissues, or individual cells. Alternatively, the sample can comprise purified or partially purified nucleic acid molecules and, for example, buffers and/or reagents that are used to generate appropriate conditions for successfully performing a method of the invention.

In one embodiment of the invention, the target nucleic acid molecules in a sample can comprise genomic DNA, genomic RNA, expressed RNA, plasmid DNA, cellular nucleic acids or nucleic acids derived from cellular organelles (e.g. mitochondria) or parasites, or a combination thereof.

In certain embodiments, the invention provides methods for detecting gene expression based on the reliable detection of a target nucleic molecule in total genomic RNA or DNA without the need for reverse transcription, PCR amplification or any other amplification method and without the need for fluorescent labeling. Specifically, the methods of the invention comprise a combination of hybridization conditions (including reaction volume, salts, formamide, temperature, and assay format), capture oligonucleotide sequences bound to a substrate, a detection probe, and a sufficiently sensitive means for detecting a target nucleic acid molecule that has been recognized by both the capture oligonucleotide and the detection probe.

As demonstrated in the Examples, the invention provides for the first time a successful method for performing gene expression analysis in total human RNA without prior amplification to selectively enrich for the target sequence, and without the aid of any enzymatic reaction, by single-step hybridization, which encompasses two hybridization events: hybridization of a first portion of the target sequence to the capture probe, and hybridization of a second portion of said target sequence to the detection probe. Both hybridization events happen in the same reaction. The target can bind to a capture oligonucleotide first and then hybridize also to a detection probe, such as the nanoparticle shown in the schematic, or the target can bind the detection probe first and then the capture oligonucleotide.

In one embodiment, the methods of the invention can be accomplished using a two-step hybridization. In this process, the hybridization events happen in two separate reactions. The target binds to the capture oligonucleotides first, and after removal of all non-bound nucleic acids, a second hybridization is performed that provides detection probes that can specifically bind to a second portion of the captured target nucleic acid.

Methods of the invention that involve the two-step hybridization will work without accommodating certain unique properties of the detection probes (such as high Tm and sharp melting behavior of nanoparticle probes) during the first hybridization event (i.e. capture of the target nucleic acid molecule) since the reaction occurs in two steps. The first step is not sufficiently stringent to capture only the desired target sequences. Thus, the second step (binding of detection probes) is then provided to achieve the desired specificity for the target nucleic acid molecule. The combination of these two discriminating hybridization events allows the overall specificity for the target nucleic acid molecule. However, in order to achieve this exquisite specificity the hybridization conditions are chosen to be very stringent. Under such stringent conditions, only a small amount of target and detection probe gets captured by the capture probes. This amount of target is typically so small that it escapes detection by standard fluorescent methods because it is buried in the background. It is therefore critical for this invention to detect this small amount of target using an appropriately designed detection probe. The detection probes described in this invention consist in a carrier portion that is typically modified to contain many detection oligonucleotides, which enhances the hybridization kinetics of this detection probe. Second, the detection probe is also labeled with one or more high sensitivity label moieties, which together with the appropriate detection instrument, allows for the detection of the small number of captured target-detection probe complexes. Thus, it is the appropriate tuning of all factors in combination with a high sensitivity detection system that allows this process to work.

The two-step hybridization methods of the invention can comprise using any detection probes as described herein for the detection step. In a preferred embodiment, nanoparticle probes are used in the second step of the method. Where nanoparticles are used and the stringency conditions in the second hybridization step are equal to those in the first step, the detection oligonucleotides on the nanoparticle probes can be longer than the capture oligonucleotides. Thus, conditions necessary for the unique features of the nanoparticle probes (high Tm and sharp melting behavior) are not needed.

The single- and two-step hybridization methods in combination with the appropriately designed capture oligonucleotides and detection probes of the invention provide new and unexpected advantages over previous methods of detecting target nucleic acid sequences in a sample. Specifically, the methods of the invention do not require an amplification step to maximize the number of targets and simultaneously reduce the relative concentration of non-target sequences in a sample to enhance the possibility of binding to the target, as required, for example, in polymerase chain reaction (PCR) based detection methods. Specific detection without prior target sequence amplification provides tremendous advantages. For example, amplification often leads to contamination of research or diagnostic labs, resulting in false positive test outcomes. PCR or other target amplifications require specifically trained personnel, costly enzymes and specialized equipment. Most importantly, the efficiency of amplification can vary with each target sequence and primer pair, leading to errors or failures in determining the target sequences and/or the relative amount of the target sequences present in a genome. In addition, the methods of the invention involve fewer steps and are thus easier and more efficient to perform than gel-based methods of detecting nucleic acid targets, such as Southern and Northern blot assays.

In one embodiment of the invention, a method is provided for detecting or quantitating gene expression in a sample, said sample believed to have one or more different types of unlabeled target nucleic acids, each type of target nucleic acid having an oligonucleotide tail, said method comprising: providing a substrate having a plurality of types of capture nucleic acid sequences attached thereto in an array for the detection of multiple portions of a target nucleic acid, the detection of multiple different target nucleic acids, or both; providing nanoparticles having oligonucleotides bound thereto, the oligonucleotides bound to the nanoparticles having a sequence that is complementary to at least a portion of the oligonucleotide tail; contacting the sample, the substrate, and the nanoparticles, said contracting occurring under conditions effective for hybridization of the target nucleic acids to the capture nucleic acid sequences bound to the substrate and hybridization of the target nucleic acids to the nanoparticles; and observing a detectable change.

In one aspect of the invention, the target nucleic acid may be RNA, e.g., mRNA, or DNA, e.g., cDNA. The oligonucleotide tail comprises a poly dT, a poly dA, or a synthetic oligonucleotide having a predetermined sequence. The oligonucleotides bound to the nanoparticles comprises a poly dT, a poly dA, or a synthetic oligonucleotide having a predetermined sequence. The capture nucleic acid sequences comprise an oligonucleotide, cDNA, or genomic sequence fragment. The nanoparticles may be made of gold.

In another aspect of the invention, the sample is first contacted with the substrate, said contacting occurring under conditions effective for hybridization of the target nucleic acids with the capture nucleic acid sequence bound to the substrate, and then contacting the target nucleic acid bound to the substrate with the nanoparticles, said contacting occurring under conditions effective for hybridization of the target nucleic acids bound to the substrate with the oligonucleotides bound to the nanoparticles.

In another aspect of the invention, the sample is first contacted with the nanoparticles, said contacting occurring under conditions effective for hybridization of the target nucleic acids with the oligonucleotides bound to the nanoparticles, and then contacting the target nucleic acid bound to the nanoparticles with the substrate, said contacting occurring under conditions effective for hybridization of the target nucleic acids bound to the nanoparticles with the capture nucleic acid sequences bound to the substrate.

In another aspect of the invention, the sample, nanoparticles and substrate are contacted simultaneously under conditions effective for hybridization of the target nucleic acids with the oligonucleotides bound to the nanoparticles and with the capture nucleic acid sequences bound to the substrate.

In another aspect of the invention, the detectable change is observed after contacting the substrate having target nucleic acids and nanoparticles with a staining material. The staining material may be silver stain or any suitable staining material.

In another aspect of the invention, a method is provided for detecting or quantitating gene expression in a sample, said sample believed to have one or more different types of unlabeled target ribonucleic acids, each type of target ribonucleic acid including a poly dA olignonucleotide tail or a synthetic oligonucleotide tail of a predetermined sequence, said method comprising: providing a substrate having a plurality of types of capture nucleic acid sequences attached thereto in an array for the detection of multiple portions of a target ribonucleic acid, the detection of multiple different target ribonucleic acids, or both; providing nanoparticles having bound thereto poly dT oligonucleotides or a synthetic oligonucleotide sequence complementary to the sequence of the oligonucleotide tail; contacting the sample, the substrate, and the nanoparticles, said contracting occurring under conditions effective for hybridization of the target ribonucleic acids to the capture nucleic acid sequences to the substrate and hybridization of the target ribonucleic acids to the nanoparticles; and contacting the nanoparticles bound to the support with a staining material to produce a detectable change; and observing the detectable change.

In one aspect of the invention, the sample is first contacted with the substrate, said contacting occurring under conditions effective for hybridization of the target ribonucleic acids with the capture nucleic acid sequences bound to the substrate, and then contacting the target ribonucleic acid bound to the substrate with the nanoparticles, said contacting occurring under conditions effective for hybridization of the target ribonucleic acids bound to the substrate with the oligonucleotides bound to the nanoparticles.

In another aspect of the invention, the sample is first contacted with the nanoparticles, said contacting occurring under conditions effective for hybridization of the target ribonucleic acids with the oligonucleotides bound to the nanoparticles, and then contacting the target ribonucleic acid bound to the nanoparticles with the substrate, said contacting occurring under conditions effective for hybridization of the target ribonucleic acids bound to the nanoparticles with the capture nucleic acid sequences bound to the substrate.

In another aspect of the invention, the sample, nanoparticles and substrate are contacted simultaneously under conditions effective for hybridization of the target nucleic acids with the oligonucleotides bound to the nanoparticles and with the capture nucleic acid sequences bound to the substrate.

In another aspect of the invention, the capture nucleic acid sequences comprise an oligonucleotide, cDNA, or genomic sequence fragment.

In another embodiment of the invention, a method is provided for detecting or quantitating gene expression in a sample, said sample believed to have one or more different types of target cDNAs, each type of target cDNA including a poly dT olignonucleotide tail or a synthetic oligonucleotide tail having a predetermined sequence, said method comprising: providing a substrate having a plurality of types of capture nucleic acid sequences attached thereto in an array for the detection of multiple portions of a target ribonucleic acid, the detection of multiple different target ribonucleic acids, or both; providing nanoparticles having bound thereto poly dA oligonucleotides or synthetic oligonucleotides having a predetermined sequence; contacting the sample, the substrate, and the nanoparticles, said contracting occurring under conditions effective for hybridization of the target cDNAs to the capture nucleic acid sequences bound to the substrate and hybridization of the target cDNAs to the nanoparticles; and contacting the nanoparticles bound to the support with a staining material to produce a detectable change; and observing the detectable change.

In one aspect of the invention, the sample is first contacted with the substrate, said contacting occurring under conditions effective for hybridization of the target cDNAs with the capture nucleic acid sequences bound to the substrate, and then contacting the target cDNAs bound to the substrate with the nanoparticles, said contacting occurring under conditions effective for hybridization of the target cDNAs bound to the substrate with the oligonucleotides bound to the nanoparticles.

In another aspect of the invention, the sample is first contacted with the nanoparticles, said contacting occurring under conditions effective for hybridization of the target cDNAs with the oligonucleotides bound to the nanoparticles, and then contacting the target cDNAs bound to the nanoparticles with the substrate, said contacting occurring under conditions effective for hybridization of the target cDNAs bound to the nanoparticles with the capture nucleic acid sequences bound to the substrate.

In another aspect of the invention, the target cDNAs, nanoparticles and substrate are contacted simultaneously under conditions effective for hybridization of the target cDNAs with the oligonucleotides bound to the nanoparticles and with the capture nucleic acid sequences bound to the substrate.

In another aspect of the invention, the capture nucleic acid sequences comprise an oligonucleotide, cDNA, or genomic sequence fragment.

In another embodiment, a detector oligonucleotide can be detectably labeled. Various methods of labeling polynucleotides are known in the art and may be used advantageously in the methods disclosed herein. In a particular embodiment, a detectable label of the invention can be fluorescent, luminescent, Raman active, phosphorescent, radioactive, or efficient in scattering light, have a unique mass, or other has some other easily and specifically detectable physical or chemical property, and in order to enhance said detectable property the label can be aggregated or can be attached in one or more copies to a carrier, such as a dendrimer, a molecular aggregate, a quantum dot, or a bead. The label can allow for detection, for example, by photonic, electronic, acoustic, opto-acoustic, gravity, electro-chemical, enzymatic, chemical, Raman, or mass-spectrometric means.

In one embodiment, a detector probe of the invention can be a nanoparticle probe having detector oligonucleotides bound thereto. Nanoparticles have been a subject of intense interest owing to their unique physical and chemical properties that stem from their size. Due to these properties, nanoparticles offer a promising pathway for the development of new types of biological sensors that are more sensitive, more specific, and more cost effective than conventional detection methods. Methods for synthesizing nanoparticles and methodologies for studying their resulting properties have been widely developed over the past 10 years (Klabunde, editor, Nanoscale Materials in Chemistry, Wiley Interscience, 2001). However, their use in biological sensing has been limited by the lack of robust methods for functionalizing nanoparticles with biological molecules of interest due to the inherent incompatibilities of these two disparate materials. A highly effective method for functionalizing nanoparticles with modified oligonucleotides has been developed. See U.S. Pat. Nos. 6,361,944 and 6,417,340 (assignee: Nanosphere, Inc.), which are incorporated by reference in their entirety. The process leads to nanoparticles that are heavily functionalized with oligonucleotides, which have surprising particle stability and hybridization properties. The resulting DNA-modified particles have also proven to be very robust as evidenced by their stability in solutions containing elevated electrolyte concentrations, stability towards centrifugation or freezing, and thermal stability when repeatedly heated and cooled. This loading process also is controllable and adaptable. Nanoparticles of differing size and composition have been functionalized, and the loading of oligonucleotide recognition sequences onto the nanoparticle can be controlled via the loading process. Suitable, but non-limiting examples of nanoparticles include those described U.S. Pat. No. 6,506,564; International Patent Application No. PCT/US02/16382; U.S. patent application Ser. No. 10/431,341 filed May 7, 2003; and International Patent Application No. PCT/US03/14100; all of which are hereby incorporated by reference in their entirety.

The aforementioned loading method for preparing DNA-modified nanoparticles, particularly DNA-modified gold nanoparticle probes, has led to the development of a new colorimetric sensing scheme for oligonucleotides. This method is based on the hybridization of two gold nanoparticle probes to two distinct regions of a DNA target of interest. Since each of the probes are functionalized with multiple oligonucleotides bearing the same sequence, the binding of the target results in the formation of target DNA/gold nanoparticle probe aggregate when sufficient target is present. The DNA target recognition results in a calorimetric transition due to the decrease in interparticle distance of the particles. This colorimetric change can be monitored optically, with a UV-vis spectrophotometer, or visually with the naked eye. In addition, the color is intensified when the solutions are concentrated onto a membrane. Therefore, a simple colorimetric transition provides evidence for the presence or absence of a specific DNA sequence. Using this assay, femtomole quantities and nanomolar concentrations of model DNA targets and polymerase chain reaction (PCR) amplified nucleic acid sequences have been detected. Importantly, it has been demonstrated that gold probe/DNA target complexes exhibit extremely sharp melting transitions which makes them highly specific labels for DNA targets. In a model system, one base insertions, deletions, or mismatches were easily detectable via the spot test based on color and temperature, or by monitoring the melting transitions of the aggregates spectrophotometrically (Storhoff et. al, J. Am. Chem. Soc., 120, 1959 (1998). See also, for instance, U.S. Pat. No. 5,506,564.

As described herein, nanoparticle probes, particularly gold nanoparticle probes, are surprising and unexpectedly suited for gene expression analysis with genomic RNA and without amplification and fluorescent labeling. First, the extremely sharp melting transitions observed in nanoparticle oligonucleotide detection probe translate to a surprising and unprecedented assay specificity that could allow for mRNA detection even in a human genomic RNA background. Second, a silver-based signal amplification procedure in a RNA microarray-based assay can further provide ultra-high sensitivity enhancement.

A nanoparticle can be detected in a method of the invention, for example, using an optical or flatbed scanner. The scanner can be linked to a computer loaded with software capable of calculating grayscale measurements, and the grayscale measurements are calculated to provide a quantitative measure of the amount of nucleic acid detected.

Suitable scanners include those used to scan documents into a computer which are capable of operating in the reflective mode (e.g., a flatbed scanner), other devices capable of performing this function or which utilize the same type of optics, any type of greyscale-sensitive measurement device, and standard scanners which have been modified to scan substrates according to the invention (e.g., a flatbed scanner modified to include a holder for the substrate) (to date, it has not been found possible to use scanners operating in the transmissive mode). The resolution of the scanner must be sufficient so that the reaction area on the substrate is larger than a single pixel of the scanner. The scanner can be used with any substrate, provided that the detectable change produced by the assay can be observed against the substrate (e.g., a gray spot, such as that produced by silver staining, can be observed against a white background, but cannot be observed against a gray background). The scanner can be a black-and-white scanner or, preferably, a color scanner.

Most preferably, the scanner is a standard color scanner of the type used to scan documents into computers. Such scanners are inexpensive and readily available commercially. For instance, an Epson Expression 636 (600×600 dpi), a UMAX Astra 1200 (300×300 dpi), or a Microtec 1600 (1600×1600 dpi) can be used. The scanner is linked to a computer loaded with software for processing the images obtained by scanning the substrate. The software can be standard software which is readily available commercially, such as Adobe Photoshop 5.2 and Corel Photopaint 8.0. Using the software to calculate greyscale measurements provides a means of quantitating the results of the assays.

The software can also provide a color number for colored spots and can generate images (e.g., printouts) of the scans, which can be reviewed to provide a qualitative determination of the presence of a nucleic acid, the quantity of a nucleic acid, or both. In addition, it has been found that the sensitivity of assays can be increased by subtracting the color that represents a negative result from the color that represents a positive result.

The computer can be a standard personal computer, which is readily available commercially. Thus, the use of a standard scanner linked to a standard computer loaded with standard software can provide a convenient, easy, inexpensive means of detecting and quantitating nucleic acids when the assays are performed on substrates. The scans can also be stored in the computer to maintain a record of the results for further reference or use. Of course, more sophisticated instruments and software can be used, if desired.

Silver staining can be employed with any type of nanoparticles that catalyze the reduction of silver. Preferred are nanoparticles made of noble metals (e.g., gold and silver). See Bassell, et al., J. Cell Biol., 126, 863-876 (1994); Braun-Howland et al., Biotechniques, 13, 928-931 (1992). If the nanoparticles being employed for the detection of a nucleic acid do not catalyze the reduction of silver, then silver ions can be complexed to the nucleic acid to catalyze the reduction. See Braun et al., Nature, 391, 775 (1998). Also, silver stains are known which can react with the phosphate groups on nucleic acids.

Silver staining can be used to produce or enhance a detectable change in any assay performed on a substrate, including those described above. In particular, silver staining has been found to provide a huge increase in sensitivity for assays employing a single type of nanoparticle so that the use of layers of nanoparticles, aggregate probes and core probes can often be eliminated.

In another embodiment, oligonucleotides attached to a substrate can be located between two electrodes, the nanoparticles can be made of a material that is a conductor of electricity, and step (d) in the methods of the invention can comprise detecting a change in conductivity. In yet another embodiment, a plurality of oligonucleotides, each of which can recognize a different target nucleic acid sequence, are attached to a substrate in an array of spots and each spot of oligonucleotides is located between two electrodes, the nanoparticles are made of a material that is a conductor of electricity, and step (d) in the methods of the invention comprises detecting a change in conductivity. The electrodes can be made, for example, of gold and the nanoparticles are made of gold. Alternatively, a substrate can be contacted with silver stain to produce a change in conductivity.

In another embodiment of the invention, a kit is provided for detecting or quantitating gene expression in a sample, said sample believed to have one or more different types of unlabeled target nucleic acids, each type of target nucleic acid including a poly dT, poly dA oligonucleotide tail, or a synthetic oligonucleotide tail having a predetermined sequence, said kit comprising: a substrate having a plurality of types of capture nucleic acid sequences attached thereto in an array for the detection of multiple portions of a target nucleic acid, the detection of multiple different target nucleic acids, or both; and one or more types of nanoparticles having bound thereto poly dT oligonucleotides, poly dA oligonucleotides, or synthetic oligonucleotides having a predetermined sequence.

### EXAMPLES

The invention is demonstrated further by the following illustrative examples. The examples are offered by way of illustration and are not intended to limit the invention in any manner. In these examples all percentages are by weight if for solids and by volume if for liquids, and all temperatures are in degrees Celsius unless otherwise noted.

### Example 1

Single-Step and Two-Step Hybridization Methods for Label-Free Gene Expression Detection Using Nanoparticle Probes

Nanoparticle-oligonucleotide probes to detect target RNA sequences were prepared using procedures described in PCT/US97/12783, filed Jul. 21, 1997; PCT/US00/17507, filed Jun. 26, 2000; PCT/US01/01190, filed Jan. 12, 2001, which are incorporated by reference in their entirety. Universal gold nanoparticle probes having oligonucleotides bound thereto were used for detection of various target RNA targets using a DNA microarray having anti-sense capture probe oligonucleotides. Nanoparticles (e.g., 15 nm gold particles) are functionalized with poly dT (6mer to 100mer) or poly dA (6mer to 100mer) oligonucleotides, or a unique oligonucleotides (6mer-100mer) via a di-sulfide bond. The poly dT- or poly dA- or unique oligonucleotides-modified gold particles serve as the universal probes for label-free expression analysis discussed in Example 2. The sequence of the oligonucleotides bound to the nanoparticles are complementary to one portion (e.g., a polyA tail) of the sequence of RNA target while the sequence of the capture oligonucleotides bound to the array glass chip are complementary to another portion of the target sequence. Under hybridization conditions, the nanoparticle probes, the capture probes, and the target sequence bind to form a complex. Signal detection of the resulting complex can be enhanced with conventional silver staining.

Part A: Materials and Methods

(a) Preparation of Gold Nanoparticles

Gold colloids (15 nm diameter) were prepared by reduction of HAuCl₄ with citrate as described in Frens, 1973, Nature Phys. Sci., 241:20 and Grabar, 1995, Anal. Chem. 67:735. Briefly, all glassware was cleaned in aqua regia (3 parts HCl, 1 part HNO₃), rinsed with Nanopure H₂O, then oven dried prior to use. HAuCl₄ and sodium citrate were purchased from Aldrich Chemical Company. Aqueous HAuCl₄ (1 mM, 500 mL) was brought to reflux while stirring. Then, 38.8 mM sodium citrate (50 mL) was added quickly. The solution color changed from pale yellow to burgundy, and refluxing was continued for 15 min. After cooling to room temperature, the red solution was filtered through a Micron Separations Inc. 1 micron filter. Au colloids were characterized by UV-vis spectroscopy using a Hewlett Packard 8452A diode array spectrophotometer and by Transmission Electron Microscopy (TEM) using a Hitachi 8100 transmission electron microscope. Gold particles with diameters of 15 nm will produce a visible color change when aggregated with target and probe oligonucleotide sequences in the 10-35 nucleotide range.

(b) Synthesis of Oligonucleotides

The capture probe oligonucleotides complementary to segments of a target RNA were synthesized on a 1 micromole scale using a ABI 8909 DNA synthesizer in single column mode using phosphoramidite chemistry [Eckstein, F. (ed.) Oligonucleotides and Analogues: A Practical Approach (IRL Press, Oxford, 1991)]. The capture sequences contained either a 3′-amino modifier that serves as the active group for covalent attachment during the arraying process. The oligonucleotides were synthesized by following standard protocols for DNA synthesis. Columns with the 3′-amino modifier attached to the solid support, the standard nucleotide phosphoramidites and reagents were obtained from Glen Research. The final dimethoxytrityl (DMT) protecting group was not cleaved from the oligonucleotides to aid in purification. After synthesis, DNA was cleaved from the solid support using aqueous ammonia, resulting in the generation of a DNA molecule containing a free amine at the 3′-end. Reverse phase HPLC was performed with an Agilent 1100 series instrument equipped with a reverse phase column (Vydac) by using 0.03 M Et₃NH⁺ OAc⁻ buffer (TEAA), pH 7, with a 1%/min. gradient of 95% CH₃CN/5% TEAA. The flow rate was 1 mL/min. with UV detection at 260 nm. After collection and evaporation of the buffer, the DMT was cleaved from the oligonucleotides by treatment with 80% acetic acid for 30 min at room temperature. The solution was then evaporated to near dryness, water was added, and the cleaved DMT was extracted from the aqueous oligonucleotide solution using ethyl acetate. The amount of oligonucleotide was determined by absorbance at 260 nm, and final purity assessed by analytical reverse phase HPLC.

The capture sequences employed in to generate the Figures are as follows:

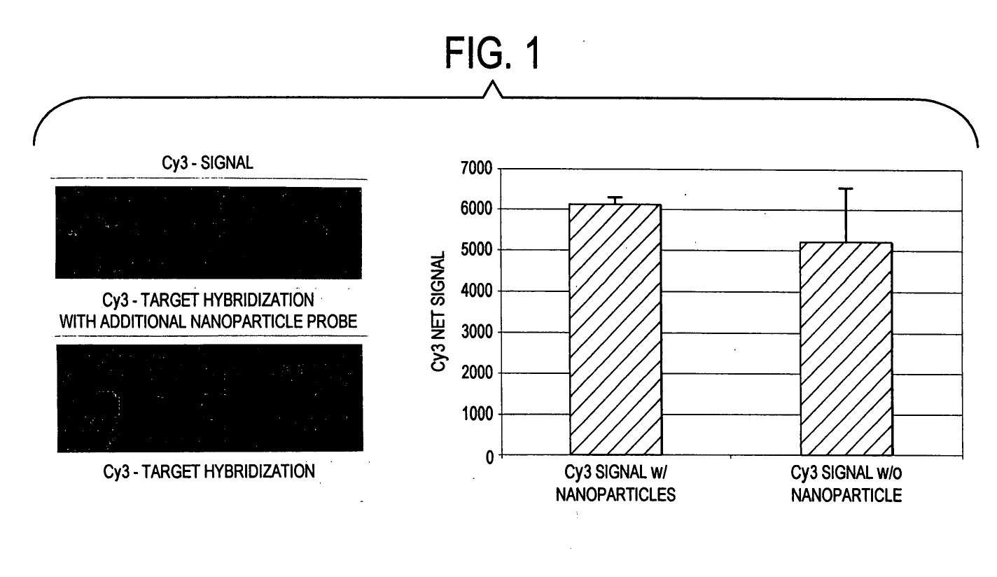

For FIG. 1:

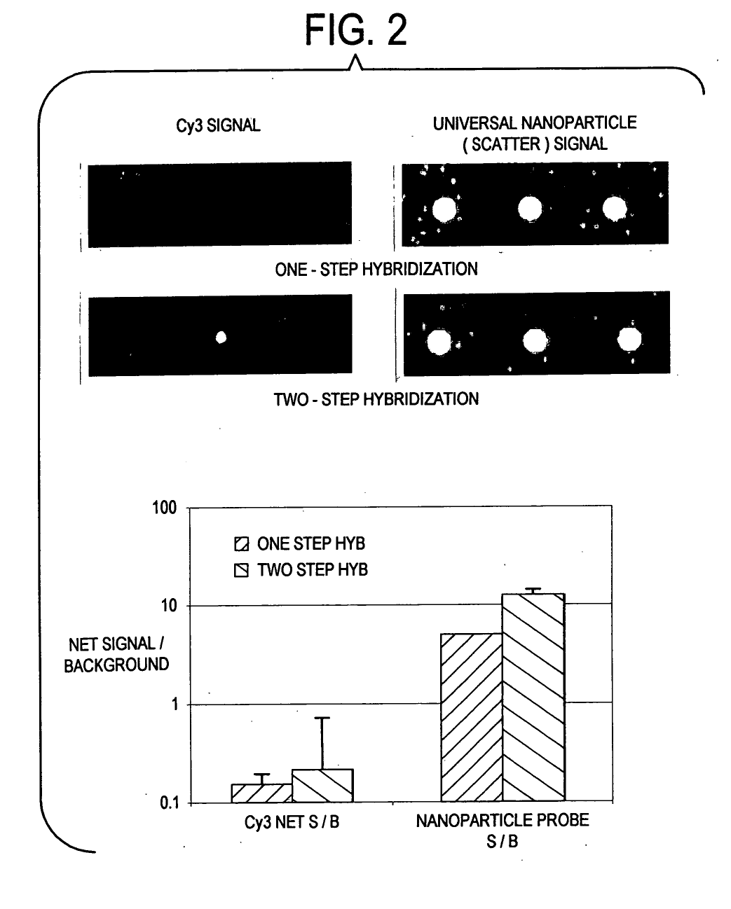

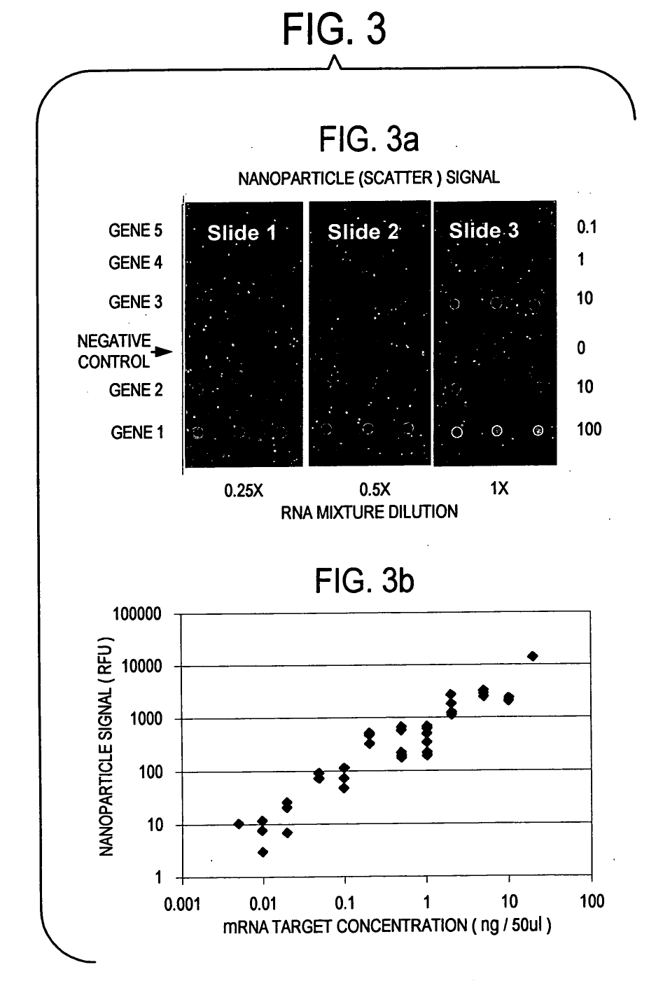

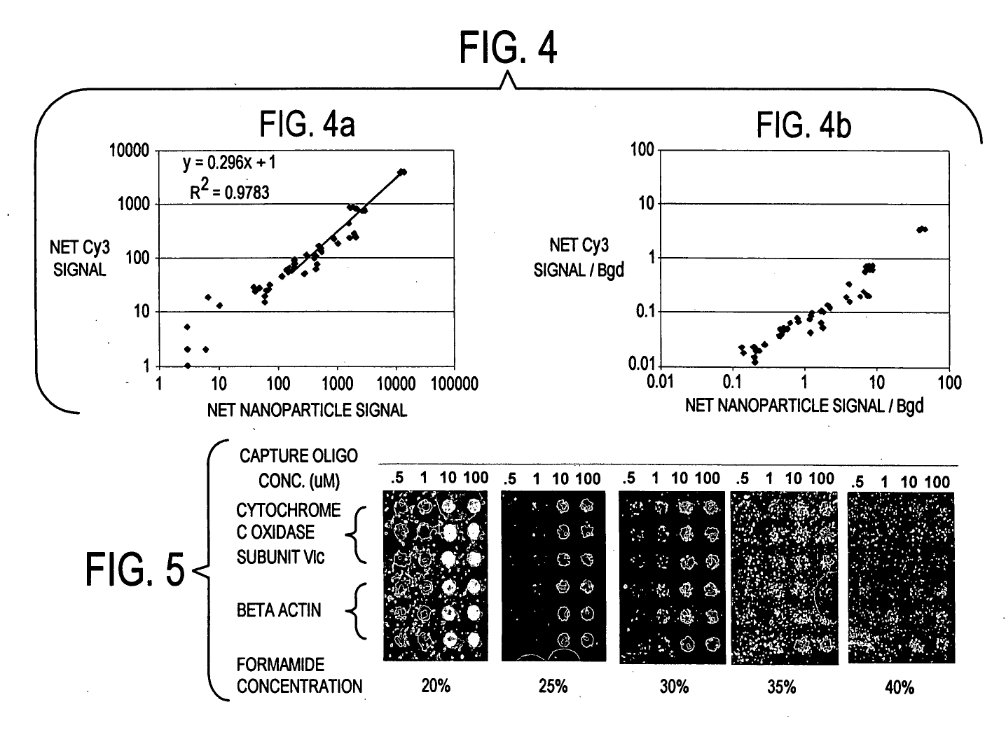

Capture oligos were AarrayControl Sense oligo Spots 1-8 (sequence not available) (Cat#1781, Ambion, Austin, Tex., USA)
   For FIG. 2:
Capture oligos were AarrayControl Sense oligo Spots 1-8 (sequence not available) (Cat#1781, Ambion, Austin, Tex., USA)
   For FIG. 3:
Capture oligos were AarrayControl Sense oligo Spots 1-8 (sequence not available) (Cat#1781, Ambion, Austin, Tex., USA)
   For FIG. 4:
Capture oligos were AarrayControl Sense oligo Spots 1-8 (sequence not available) (Cat#1781, Ambion, Austin, Tex., USA)

For FIG. 5:

cytochrome c oxidase subunit Vic sense oligo
5′-NH2-ctgtttgtcactgggtgacctcccgtccttgtgggcgctccac
gggccctggtctacgggccttcatgag-3′

Beta actin sense oligo
5′-NH2-tggaacggtgaaggtgacagcagtcggttggagcgagcatccc
ccaaagttcacaatgtggccgaggact-3

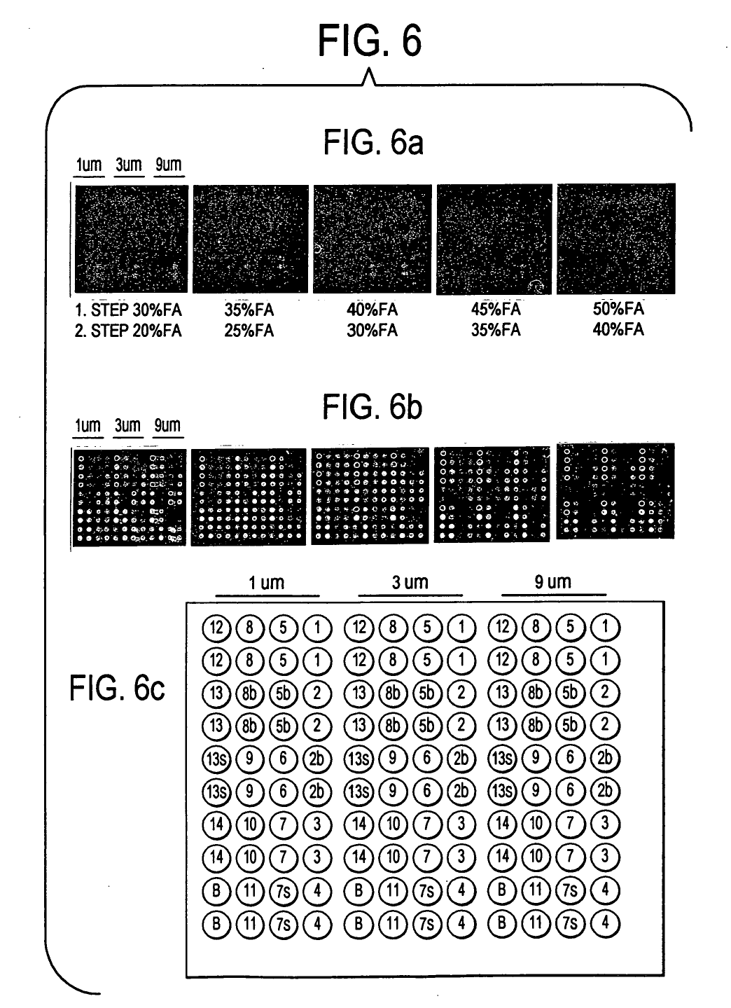

For FIG. 6:

Capture Sequences:

1 (glutamyl-prolyl-tRNA synthetase:
gagggtttccaggtttatattcctggccagttttctccttatattcagc
t-NH2

2 (Homo sapiens cDNA clone IMAGE: 4093756, partial
cds):
acacatccgtctcctctgcgatataaccaaatggtgtttgacggttgaa
t-NH2
2B:
ttaatgtttctaacaaagcgtatcatgcaaacggagattagaggttata
c-NH2

3 (hypothetical protein FLJ14668):
taagggagtcagctcatcctagcccaagttgcttactttttctcccttg
a-NH2

4 (3-ketoacyl CoA thiolase beta-subunit of
mitochondrial trifunctional protein, exon 8, 9,
10):
ccgtagggcttgatgaatgcaggttttagtttggccatctgctccagtg
a-NH2

5 (chromatin assembly factor 1, subunit B (p60)):
tgtgtgcactttcacgaggatgccagggaggactcactgattttcacac
t-NH2
5B:
atactctaaaattcgacagagtaaaatctcaaattactttctcatcttc
c-NH2

6 (transcription factor 3, TCF3):
actgctgtttcttcctcctcgcgctgggtgaatctcgtttgaattctat
g-NH2

7 (cDNA FLJ37123 fis):
cggaagttggaggcgtcatgcagcgcctcctgcctgggagccaggcgat
c-NH2

7S:
NH2-atcgcctggctcccaggcaggaggcgctgcatgacgcctccaactt
ccg

8 (adenosine monophosphate deaminase 2, isoform
L):
aacaccactcccggggttgagtggcagatccaggactttgcagcaactg
t-NH2
8B:
tatgaaacactgcagttcacagcaaaggcctcagtccagaacacaacat
a-NH2

9 (chromatin assembly factor 1, subunit B (p60)):
tgtgtgcactttcacgaggatgccagggaggactcactgattttcacac
t-NH2

10 (isoleucine-tRNA synthetase):
tgtaacctgctcccaacatgactgcataggtgtctaaggttaagtgtga
a-NH2

11 (seryl-tRNA synthetase):
tggtttcatcagtcatcaatgatgggtccctatgcccatgcgaggagac
a-NH2

12 (Ribosomal Protein L32):
tactcattttcttcactgcgcagcctggcattggggttggtgactctga
t-NH2

13 (actin, beta):
actgggccattctccttagagagaagtggggtggcttttaggatggcaa
g-NH2

13S:
NH2-ttgccatcctaaaagccaccccacttctctctaaggagaatggccc
agt

14 (ubiquitin B):
atcttggccttcacattttcgatggtgtcactgggctccacctccagag
t-NH2

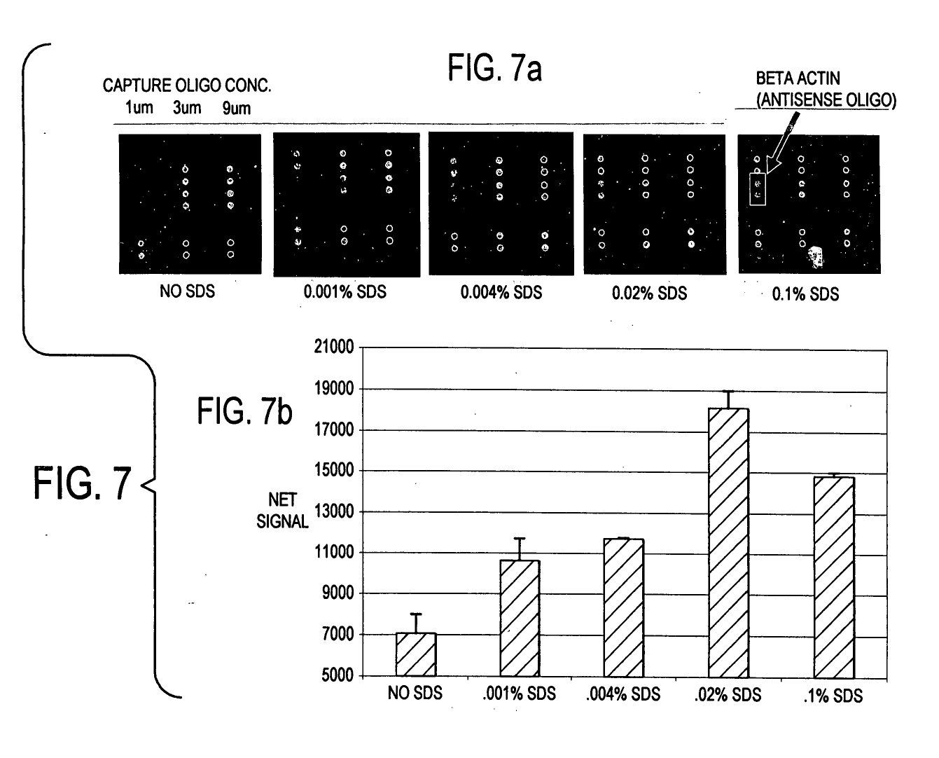

For FIG. 7:

Capture Sequences:

1 (glutamyl-prolyl-tRNA synthetase):
gagggtttccaggtttatattcctggccagttttctccttatattcagc
t-NH2

2 (Homo sapiens cDNA clone IMAGE: 4093756, partial
cds):
acacatccgtctcctctgcgatataaccaaatggtgtttgacggttgaa
t-NH2
2B:
ttaatgtttctaacaaagcgtatcatgcaaacggagattagaggttata
c-NH2

3 (hypothetical protein FLJ1668):
taagggagtcagctcatcctagcccaagttgcttactttttctcccttg
a-NH2

4 (3-ketoacyl CoA thiolase beta-subunit of
mitochondrial trifunctional protein, exon 8, 9,
10):
ccgtagggcttgatgaatgcaggttttagtttggccatctgctccagtg
a-NH2

5 (chromatin assembly factor 1, subunit B (p60)):
tgtgtgcactttcacgaggatgccagggaggactcactgattttcacac
t-NH2
5B:
atactctaaaattcgacagagtaaaatctcaaattactttctcatcttc
c-NH2

6 (transcription factor 3, TCF3):
actgctgtttcttcctcctcgcgctgggtgaatctcgtttgaattctat
g-NH2

7 (cDNA FLJ37123 fis):
cggaagttggaggcgtcatgcagcgcctcctgcctgggagccaggcgat
c-NH2

7S:
NH2-atcgcctggctcccaggcaggaggcgctgcatgacgcctccaact
tccg

8 (adenosine monophosphate deaminase 2, isoform
L):
aacaccactcccggggttgagtggcagatccaggactttgcagcaactg
t-NH2
8B:
tatgaaacactgcagttcacagcaaaggcctcagtccagaacacaacat
a-NH2

9 (chromatin assembly factor 1, subunit B (p60)):
tgtgtgcactttcacgaggatgccagggaggactcactgattttcacac
t-NH2

10 (isoleucine-tRNA synthetase):
tgtaacctgctcccaacatgactgcataggtgtctaaggttaagtgtga
a-NH2

11 (seryl-tRNA synthetase):
tggtttcatcagtcatcaatgatgggtccctatgcccatgcgaggagac
a-NH2

12 (Ribosomal Protein L32):
tactcattttcttcactgcgcagcctggcattggggttggtgactctga
t-NH2

13 (actin, beta):
actgggccattctccttagagagaagtggggtggcttttaggatggcaa
g-NH2

13S:
NH2-ttgccatcctaaaagccaccccacttctctctaaggagaatggccc
agt

14 (ubiquitin B):
atcttggccttcacattttcgatggtgtcactgggctccacctccagag
t-NH2

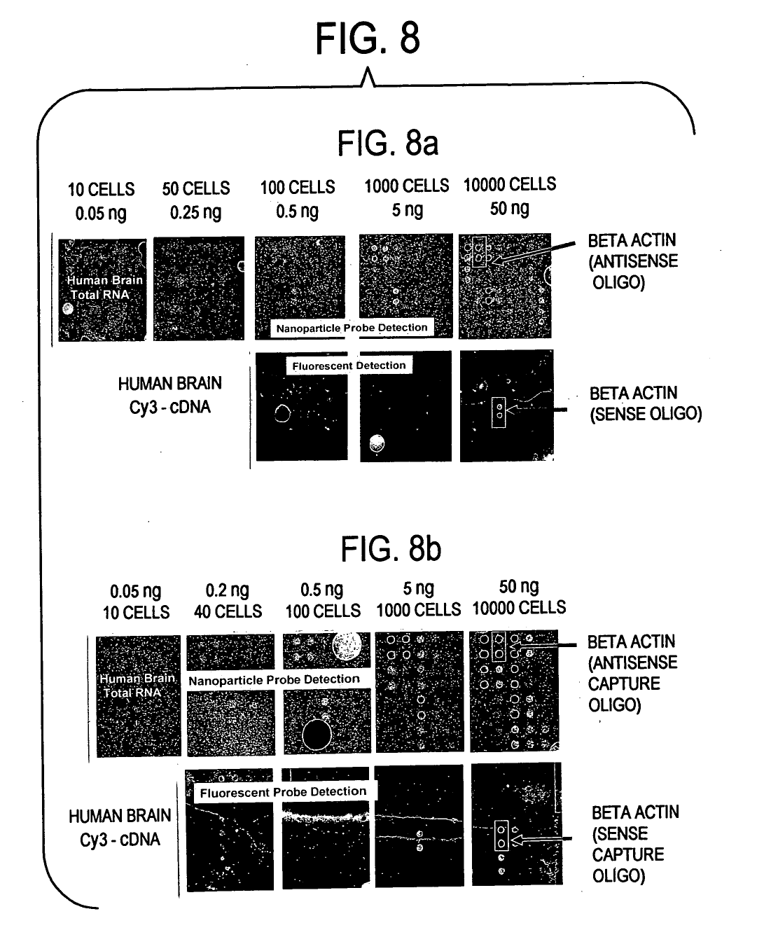

For FIG. 8:

Capture Sequences

1 (glutamyl-prolyl-tRNA synthetase):
gagggtttccaggtttatattcctggccagttttctccttatattcagc
t-NH2

2 (Homo sapiens cDNA clone IMAGE: 4093756, partial
cds):
acacatccgtctcctctgcgatataaccaaatggtgtttgacggttgaa
t-NH2
2B:
ttaatgtttctaacaaagcgtatcatgcaaacggagattagaggttata
c-NH2

3 (hypothetical protein FLJ14668):
taagggagtcagctcatcctagcccaagttgcttactttttctcccttg
a-NH2

4 (3-hetoacyl CoA thiolase beta-subunit of
mitochondrial trifunctional protein, exon 8, 9,
10):
ccgtagggcttgatgaatgcaggttttagtttggccatctgctccagtg
a-NH2

5 (chromatin assembly factor 1, subunit B (p60)):
tgtgtgcactttcacgaggatgccagggaggactcactgattttcacac
t-NH2
5B:
atactctaaaattcgacagagtaaaatctcaaattactttctcatcttc
c-NH2

6 (transcription factor 3, TCF3):
actgctgtttcttcctcctcgcgctgggtgaatctcgtttgaattctat
g-NH2

7 (cDNA FLJ37123 fis):
cggaagttggaggcgtcatgcagcgcctcctgcctgggagccaggcgat
c-NH2

7S:
NH2-atcgcctggctcccaggcaggaggcgctgcatgacgcctccaactt
ccg

8 (adenosine monphosphate deaminase 2, isoform L):
aacaccactcccggggttgagtggcagatccaggactttgcagcaactg
t-NH2
8B:
tatgaaacactgcagttcacagcaaaggcctcagtccagaacacaacat
a-NH2

9 (chromatin assembly factor 1, subunity B (p60)):
tgtgtgcactttcacgaggatgccagggaggactcactgattttcacac
t-NH2

10 (isoleucine-tRNA synthetase):
tgtaacctgctcccaacatgactgcataggtgtctaaggttaagtgtga
a-NH2

11 (seryl-tRNA synthetase):
tggtttcatcagtcatcaatgatgggtccctatgcccatgcgaggagac
a-NH2

12 (Ribosomal Protein L32):
tactcattttcttcactgcgcagcctggcattggggttggtgactctga
t-NH2

13 (actin, beta):
actgggccattctccttagagagaagtggggtggcttttaggatggcaa
g-NH2

13S:
NH2-ttgccatcctaaaagccaccccacttctctctaaggagaatggccc
agt

14 (ubiquitin B):
atcttggccttcacattttcgatggtgtcactgggctccacctccagag
t-NH2

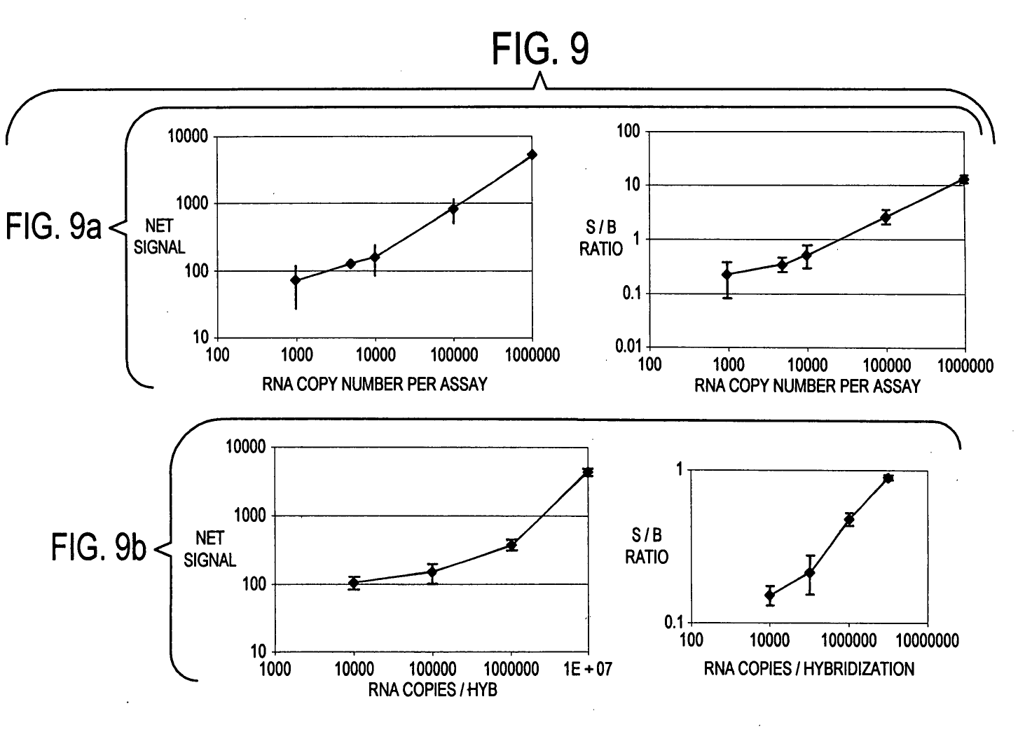

For FIG. 9:
Capture oligos were AarrayControl Sense oligo Spots 1-8 (sequence not available) (Cat#1781, Ambion, Austin, Tex., USA)

The detection probe oligonucleotides designed to detect target RNA sequences comprise a steroid disulfide linker at the 5′-end followed by the recognition sequence. The sequences for the probes are described:

For FIG. 1:
5′-S-aaaaaaaaaaaaaaaaaaaA-3′
For FIG. 2:
5′-S-aaaaaaaaaaaaaaaaaaaa-3′
For FIG. 3:
5′-S-aaaaaaaaaaaaaaaaaaaa-3′
For FIG. 4:
5′-S-aaaaaaaaaaaaaaaaaaaa-3′
For FIG. 5:
5′-S-tttttttttttttttttttt-3′
For FIG. 6:
5′-S-tttttttttttttttttttt-3′
For FIG. 7:
5′-S-tttttttttttttttttttt-3′
For FIG. 8:
5′-S-tttttttttttttttttttt-3′
For FIG. 9:
5′-S-tttttttttttttttttttt-3′

S indicates a connecting unit prepared via an epiandrosterone disulfide group;

The synthesis of the detection probe oligonucleotides followed the methods described for the capture probes with the following modifications. First, instead of the amino-modifier columns, supports with the appropriate nucleotides reflecting the 3′-end of the recognition sequence were employed. Second, the 5′-terminal steroid-cyclic disulfide was introduced in a coupling step by employing a modified phosphoramidite containing the steroid disulfide (see Letsinger et al., 2000, Bioconjugate Chem. 11:289-291 and PCT/US01/01190 (Nanosphere, Inc.), the disclosure of which is incorporated by reference in its entirety). The phosphoramidite reagent may be prepared as follows: a solution of epiandrosterone (0.5 g), 1,2-dithiane-4,5-diol (0.28 g), and p-toluenesulfonic acid (15 mg) in toluene (30 mL) was refluxed for 7 h under conditions for removal of water (Dean Stark apparatus); then the toluene was removed under reduced pressure and the residue taken up in ethyl acetate. This solution was washed with water, dried over sodium sulfate, and concentrated to a syrupy residue, which on standing overnight in pentane/ether afforded a steroid-dithioketal compound as a white solid (400 mg); Rf (TLC, silica plate, ether as eluent) 0.5; for comparison, Rf values for epiandrosterone and 1,2-dithiane-4,5-diol obtained under the same conditions are 0.4, and 0.3, respectively. Recrystallization from pentane/ether afforded a white powder, mp 110-112° C.; ¹H NMR, δ 3.6 (1H, C³OH), 3.54-3.39 (2H, m 2OCH of the dithiane ring), 3.2-3.0 (4H, m 2CH₂S), 2.1-0.7 (29H, m steroid H); mass spectrum (ES⁺) calcd for C₂₃H₃₆O₃S₂ (M+H) 425.2179, found 425.2151. Anal. (C₂₃H₃₇O₃S₂) S: calcd, 15.12; found, 15.26. To prepare the steroid-disulfide ketal phosphoramidite derivative, the steroid-dithioketal (100 mg) was dissolved in THF (3 mL) and cooled in a dry ice alcohol bath. N,N-diisopropylethylamine (80 μL) and β-cyanoethyl chlorodiisopropylphosphoramidite (80 μL) were added successively; then the mixture was warmed to room temperature, stirred for 2 h, mixed with ethyl acetate (100 mL), washed with 5% aq. NaHCO₃ and with water, dried over sodium sulfate, and concentrated to dryness. The residue was taken up in the minimum amount of dichloromethane, precipitated at −70° C. by addition of hexane, and dried under vacuum; yield 100 mg; ³¹p NMR 146.02. After completion of the DNA synthesis, the epiandrosterone-disulfide linked oligonucleotides were deprotected from the support under aqueous ammonia conditions and purified on HPLC using reverse phase column as described above.

(c) Attachment of Oligonucleotides to Gold Nanoparticles

The probe was prepared by incubating initially a 4 μM solution of the oligonucleotide with a ˜14 nM solution of a 15 nm citrate-stabilized gold nanoparticle colloid solution in a final volume of 2 mL for 24 h. The salt concentration in this preparation was raised gradually to 0.8 M over a period of 40 h at room temperature. The resulting solution was passed through a 0.2 μm cellulose acetate filter and the nanoparticle probe was pelleted by spinning at 13,000 G for 20 min. After removing the supernatant, the pellet was re-suspended in water. In a final step, the probe solution was pelleted again and resuspended in a probe storage buffer (10 mM phos, 100 mM NaCl, 0.01% w/v NaN₃). The concentration was adjusted to 10 nM after estimating the concentration based on the absorbance at 520 nm (ε=2.4×10⁸ M⁻¹ cm⁻¹).

| | |
|---|---|
| FIG. 1 Probe: | gold-S′-5′-aaaaaaaaaaaaaaaaaaaa-3′ |
| FIG. 2 Probe: | gold-S′-5′-aaaaaaaaaaaaaaaaaaaa-3′ |
| FIG. 3 Probe: | gold-S′-5′-aaaaaaaaaaaaaaaaaaaa-3′ |
| FIG. 4 Probe: | gold-S′-5′-aaaaaaaaaaaaaaaaaaaa-3′ |
| FIG. 5 Probe: | gold-S′-5′-tttttttttttttttttttt-3′ |
| FIG. 6 Probe: | gold-S′-5′-tttttttttttttttttttt-3′ |
| FIG. 7 Probe: | gold-S′-5′-tttttttttttttttttttt-3′ |
| FIG. 8 Probe: | gold-S′-5′-tttttttttttttttttttt-3′ |
| FIG. 9 Probe: | gold-S′-5′-tttttttttttttttttttt-3′ |

S′ indicates a connecting unit prepared via an epiandrosterone disulfide group;
(d) Preparation of DNA Microarrays

Capture strands were arrayed on CodeLinke slides (Amersham, Inc.) by using a GMS417 arrayer (Affymetrix). The positioning of the arrayed spots was designed to allow multiple hybridization experiments on each slide, achieved by partitioning the slide into separate test wells by silicone gaskets (Grace Biolabs). The spots were spotted in triplicate in manufacturer-provided spotting buffers. Protocols recommended by the manufacturer were followed for post-array processing of the slides.

(e) Hybridization Assay Conditions

In an assay, RNA targets (un-labeled total RNA or mRNA) hybridize to an array printed with anti-sense capture oligonucleotides. The poly dT-modified gold particles hybridize to the poly A tail of the mRNA molecules. Alternatively, RNA targets can be converted into cDNAs with poly dT primers or chimeric oligonucleotide primers which are composed with random primers (or poly dT primers) plus the complementary sequence to the unique oligonucleotides and are then hybridized to an array printed with sense capture oligonucleotides. The poly dA modified gold particles will hybridize to the poly dT tail of the cDNA and serve as universal probes. Alternatively, a unique oligonucleotide modified gold particles will hybridize to the complementary tail of the cDNA and serve as universal probes. The sandwich assay can be carried out in a one-step hybridization (i.e., RNA targets and oligo-modified nanoparticles are mixed together and hybridized to the array) or in a two-step hybridization assay (i.e., RNA targets are hybridized to the array first, followed by a second hybridization step with nanoparticle probes). After hybridization and removal of unbound nanoparticle probes, the bound nanoparticle probes are amplified with silver and the hybridization signal is acquired by light scattering detection. In comparison studies with fluorescent label-based detection discussed in this Example below and in Example 2, parameters such as the dynamic range of the signal, the specificity and sensitivity of the assay, and the correlation between target concentration and signal intensity are examined to evaluate the performance of label-free gene expression analysis with universal nanoparticle probes.

Part B: Feasibility Study of Universal Nanoparticle Probes for Label-Free Gene Expression Detection

A test array was designed to demonstrate the feasibility of applying universal nanoparticle probes for label-free gene expression analysis. Six gene specific oligonucleotides (amino-modified 70mer) were purchased from Ambion (Austin, Tex.) and printed in triplicate on CodeLink glass slides as discussed in Part A (above). The RNA 1-6 were purchased from Ambion (Austin, Tex.) (Cat # 1780 ArrayControl RNA Spikes 1-8). Sequences are not available.

15 nm gold nanoparticles were functionalized with poly dA (20mer) (5′-aaaaaaaaaaaaaaaaaaaa-3′). In order to compare the fluorescent signal and the universal nanoparticle probe signal, the corresponding 6 RNA targets (each about 1 Kb in length and each containing a 30mer poly A tail) were reverse transcribed with poly dT primer (18mer) in the presence of Cy3- or Cy5-labeled nucleotides purchased from Amersham (Piscataway, N.J., USA) using the procedure recommended by the manufacturer. The reverse transcription was carried out by mixing different amounts of the 6 RNA targets to generate a target concentration gradient. For example, in one tube, 100 ng RNA-1, 10 ng RNA-2, 10 ng RNA-3, 1 ng RNA-4, 0.1 ng RNA-5 and 0 ng RNA-6 were mixed together and labeled with Cy3. In another tube, 0.1 ng RNA-1, 1 ng RNA-2, 10 ng RNA-3, 10 ng RNA-4, 100 ng RNA-5 and 0 ng RNA-6 were mixed together and labeled with Cy5. The purified labeled targets of each tube were diluted in final volume of 50 ul.

1. Effect of Nanoparticle Probes on Target RNA Hybridization

To examine the effect of the presence of nanoparticle probe on target hybridization the Cy3-labeled targets were hybridized to the test array in the presence or absence of nanoparticle poly dA-probe. Gene specific RNA targets and corresponding capture oligos were purchased from Ambion (sequence information is not available). Capture oligos (30 uM) were spotted on CodeLink slides. Cys3-labeled cDNA targets were hybridized via a one-step hybridization on the microassay plate with or without the oligo-dA 20mer gold particle probe in a mixture containing 30% formamide, 5×SSC, and 0.05% Tween 20 at 40° C. for one hour. After hybridization, the array was washed in 0.5M NaNO3 and 0.05% Tween 20 at room temperature for 2 minutes (twice), in 2×SCC at room temperature for 2 minutes, and then in 0.5×SSC for 0 seconds. After spin-drying the slide, the slide was imaged with an imaging device arrayWoRX (Model No.e, Applied Precision, Inc. Issaquah, Wash., USA) at the Cy3 channel.

After hybridization, the Cy3 signal was found to be similar for both conditions (presence or absence of nanoparticles), indicating that there is no negative interference of nanoparticle probes on target hybridization (FIG. 1). These results also suggested that a one-step sandwich hybridization can be performed with nanoparticle probes.

2. Comparison of Sensitivity of Nanoparticle Label and Fluorescent Label

For comparison of sensitivity, both the Cy3 signal and the nanoparticle (scatter) signal were measured for each spot on the array after hybridization with target and nanoparticle probe. A one-step and two-step hybridization assays were employed.

In the one-step hybridization assay, Cy3-labeled cDNA targets and oligomer dA 20 mer gold nanoparticle probes were co-hybridized on microarray plates in a mixture containing 30% formamide, 5×SSC, and 0.05% Tween 20 at 40° C. for 1 hour. After hybridization, the array was washed in 0.5 M NaNO₃ and 0.05% Tween 20 at room temperature for 2 minutes (twice), in 2×SSC at room temperature for two minutes, and then in 0.5×SSC for 10 seconds. After spin dry, the slide was imaged with Arraywork at Cy3 channel. The slide was further washed with 0.5M NaNO₃, and then subjected to silver stain to obtain scatter signals.

In a two-step hybridization, Cy3-labeled cDNA targets were hybridized on microarray in a mixture containing 30% formamide, 5×SCC, and 0.05% Tween 20 at 40° C. for 1 hour. After hybridization, the array was washed in 0.5M NaNO₃ and and 0.05% Tween 20 at room temperature for 2 minutes (twice), in 2×SSC at room temperature for two minutes, and then in 0.5×SSC for 10 seconds. After spin drying, the slide was imaged with Arraywork at Cy3 channel. The slide was then hybridized with oligo dA-20mer gold particle probe in a mixture containing 30% formamide, 5×SCC, and 0.05% Tween 20 at 40° C. for 45 minutes. After probe hybridization step, the slide was washed with 0.5M NaNO₃ and 0.05% Tween 20 at room temperature for 2 minutes (twice), and then the slide was subjected to silver stain to obtain scatter signals. The nanoparticle scatter signal was measured using a an arrayWoRx device (model. No. e, Applied Precision, Issaquah, Wash., USA. The net signal (raw signal minus local background) to background ratio was found to be 30-50 fold greater for the nanoparticle probe than the corresponding Cy3-fluorescent signal, independent on the hybridization format, i.e. one-step or two-step hybridization (FIG. 2).

3. Determination of Correlation of Target Concentration and Nanoparticle Signal

The hybridization of universal probe is very specific and a linear correlation was observed between target concentration and nanoparticle signals over a greater than 3 log range. A target titration experiment was performed by hybridizing 3 slides with either 2.5 ul, 5 ul or 10 ul of the Cy3-labeled target mixture in the presence of the universal nanoparticle probe. Gene specific RNA targets and corresponding capture oligos were purchased from Ambion (sequence information is not available). Capture oligos (30 uM) were spotted on CodeLink slides.

In a one-step hybridization assay, RNA targets were mixed at different concentrations as indicated in FIG. 3a and then labeled with Cy3. The Cy3-labeled cDNA targets and oligomer dA 20 mer gold nanoparticle probes were co-hybridized on microarray plates in a mixture containing 30% formamide, 5×SSC, and 0.05% Tween 20 at 40° C. for 1 hour. After hybridization, the array was washed in 0.5 M NaNO3 and 0.05% Tween 20 at room temperature for 2 minutes (twice), in 2×SSC at room temperature for two minutes, and then in 0.5×SSC for 10 seconds. After spin dry, the slide was imaged with arrayWoRx device at Cy3 channel. The slide was further washed with 0.5M NaNO3, and then subjected to silver stain to obtain scatter signals.

As shown in FIGS. 3a and b, the observed scatter signal correlates with target concentration. First, there is no detectable hybridization signal on the negative control gene (gene 6, no target was added to the hybridization mix), demonstrating the specificity of hybridization. When plotting the scatter signal for all spots (3 slides) a linear correlation between target concentration and nanoparticle concentration can be demonstrated over a range of 3 logs (FIG. 3b). This result demonstrates the feasibility of using nanoparticle for detection of gene expression.

Good correlation was observed between the scatter (nanoparticle) signal and the fluorescent signal. Gene specific RNA targets and corresponding capture oligos were purchased from Ambion (sequence information is not available). Capture oligos (30 uM) were spotted on CodeLink slides. In a one-step hybridization, the RNAs were mixed at different concentrations as indicated in FIG. 3a and then labeled with Cy3. The Cy3-labeled cDNA targets and oligo-dA 20mer gold particle probe were co-hybridized on microarrays in a mixture containing 30% formamide, 5×SSC, and 0.05% TWEEN 20 at 40° C. for 1 hour. After hybridization, the array was washed in 0.5M NaNO3 and 0.05% TWEEN 20 at room temperature for 2 min (2×), in 2×SSC at room temperature for 2 min, and then in 0.5×SSC for 10 seconds. After spin drying, the slide was imaged with Arraywork at Cy3 channel. The slide was further washed with 0.5M NaNO3, and then subjected to silver stain to obtain scatter signal.

FIG. 4a shows a linear correlation between the nanoparticle signals and Cy3-fluorescent signals for a target concentration range exceeding 3 logs. However, the signal to background ratio for the scatter signal was 10-40 fold higher than that for the Cy3-fluorescent signal at all target concentrations (FIG. 4b), demonstrating the higher sensitivity of nanoparticle labels in this assay system.

### Example 2

Label-Free Human Gene Expression Analysis Using Universal Nanoparticle Probes

A human test array was designed to examine the feasibility of applying universal nanoparticle probes for label-free human gene expression analysis using the procedures and materials described in Example 1. Fourteen human gene specific oligonucleotides (amino-modified 70mer) were purchased from Midland, Tex. and printed at 100 μM on CodeLink glass slides. 15 nm gold particles were functionalized with poly dT (20mer). However, the probe-capture binding was observed with some of the capture oligonucleotides (e.g., human beta actin). The hybridization conditions are as follows: Two capture oligos (beta actin and XX) were spotted on CodeLink slides. The oligo-dT 20mer gold particle probe (1 nM) was added on to microarray in a mixture containing 20%-40% formamide, 4×SSC, and 0.04% TWEEN 20, at 40° C. for 30 min. After hybridization, the arrays were washed in 0.5M NaNO₃ and 0.05% TWEEN 20 at room temperature for 2 min (4×), and 0.5M NaNO₃ at room temperature for 2 minutes (2×). The slides were subjected to silver stain (5.5 min) to obtain scatter signal.

The nanoparticle probe and capture oligonucleotides interactions could lead to false positive signals, thus affect the accuracy of gene expression profiling. To overcome the probe-capture interaction, human beta actin capture oligomer was printed on Codelink slides (Amersham) at different concentrations (100 uM, 10 uM, 1 uM and 0.5 uM). After incubation with oligo-dT20mer modified gold nanoparticles it was found that as capture oligo concentration decreased, the probe-capture interaction is significantly reduced (FIG. 5). The reduction of nonspecific signals is also observed with increasing amounts of formamide in the hybridization mixture which lowers the TM of nucleic acid duplexes.

A human test array (amino-modified 50mer) was printed on CodeLink glass slides at different oligo concentrations (1 uM, 3 uM and 9 uM). The human test array were hybridized with total human RNA or without RNA sample as control, and then hybridized with oligo-dT20mer modified gold nanoparticles. In the hybridization and wash buffer, a combination of detergents (SDS and Tween 20) was introduced. No probe-capture interaction for all three capture oligo concentrations (FIG. 6a) was observed. The experimental conditions were as follows: Capture oligos were spotted on CodeLink slides. The oligo-dT 20mer gold particle probe (1 nM) was added on to the microarray in a mixture containing 20%-40% formamide, 4×SSC, 0.04% Tween, 0.02% SDS, at 40° C. for 30 min. After hybridization, the arrays were washed in 0.5 M NaNO₃/0.02% Tween/0.01% SDS (3×) at RT, 0.5 M NaNO₃ (2×). The slides were subjected to silver stain (5.5 min) to obtain scatter signal. The non-specific binding was not observed even at low formamide concentrations.

In addition, the human total RNA hybridization showed that the signal intensities are comparable for all three capture concentrations (FIG. 6b). Capture oligos were spotted on CodeLink slides. 1 ug of human total reference RNA was hybridized on a microarray in a mixture containing 30%-50% formamide, 4×SSC, 0.04% Tween, 0.02% SDS, at 40° C. for 2 h. After hybridization, the arrays were washed in 0.5 M NaNO₃/0.02% Tween/0.01% SDS (3×) at RT, 0.2×SSC, 10 seconds (2×), and spin dry. The arrays were further hybridized with 1 nM dT 20mer-gold nanoparticle probe in a mixture containing 20%-40% formamide, 4×SSC, 0.04% Tween, 0.02% SDS, at 40° C. for 30 min. The arrays were washed in 0.5 M NaNO₃/0.02% Tween/0.01% SDS (3×) at RT, 0.5 M NaNO₃ (2×). The slides were subjected to silver stain (5.5 min) to obtain scatter signal.

The effect of different detergent combinations on hybridization was then tested. Human test arrays were hybridized with 0.1 ug of total human universal reference RNA (BD Bioscience Clontech) with fixed Tween 20 concentration (0.04% in hybridization mixture) and titrated SDS concentrations from 0.001% to 0.1%. The hybridization signal was higher as the SDS concentration increased from 0.001% to 0.02% (FIG. 7). The experimental conditions were as follows: Capture oligos (at 1 uM, 3 uM and 9 uM) were spotted on CodeLink slides. 0.1 ug of human total reference RNA was hybridized on a microarray in a 5 ul of mixture containing 50% formamide, 4×SSC, 0.04% Tween, and different SDS concentrations as indicated, at 40° C. for 1.5 h. After hybridization, the arrays were washed in 0.5 M NaNO₃/0.02% Tween/0.001% SDS (3×) at RT, 0.2×SSC, 10 seconds (2×), and spin dry. The arrays were further hybridized with 1 nM of dT 20mer-gold nanoparticle probe in a mixture containing 30% formamide, 4×SSC, 0.04% Tween, and different SDS concentrations as indicated, at 40° C. for 30 min. The arrays were washed in 0.5 M NaNO₃/0.02% Tween/0.001% SDS (3×) at RT, 0.5 M NaNO₃ (2×). The slides were subjected to silver stain (5.5 min) to obtain scatter signal. The increased hybridization signal may be due to the reduced non-specific RNA target binding to the slide surface which results in improved hybridization kinetics.

Under improved assay conditions, the sensitivity of fluorescent based detection and the gold-nanoparticle based detection with human gene test arrays was compared. The human test arrays were printed with both human beta actin antisense oligonucleotides for direct mRNA detection with gold-nanoparticle probes and human beta actin sense oligonucleotides for Cy3-labeled cDNA detection. Human total RNA was reverse transcribed in the presence of Cy3 labeled dCTP and the resulting cDNA was hybridized to the human capture microarrays under the same conditions as was used for directly hybridizing total human brain RNA (for nanoparticle detection). To compare the sensitivity, the amount of total human brain RNA (BD Bioscience Clontech) or reverse transcripted cDNA sample was titrated down from 50 ng to 0.05 ng for each well. For a 2-hour hybridization, the specific hybridization signal on beta actin spots was observed at 0.5 ng of total human RNA with nanoparticle detection and at 50 ng of Cy3-labeled cDNA with fluorescent detection (FIG. 8a). A two-hour target hybridization was performed in the presence of 46% FM, 4×SSC/0.04% TW20, 0.01% SDS in a 5 ul reaction. Slide was washed 3 times with 0.5N NaNO₃/0.02% TW20/0.01% SDS and twice with 0.2×SSC followed by probe hybridization (32% FM, 4×SSC/0.04% TW20/0.005% SDS) for 25 min. The slide was again washed 3 times with 0.5N NaNO₃/0.02% TW20/0.005% SDS and twice with 0.5N NaNO₃ followed by silver development and light scattering detection.

For the overnight hybridization, the specific hybridization signal on beta actin spots was observed at 0.2 ng of total human RNA with nanoparticle detection and at 5 ng of Cy3-labeled cDNA with fluorescent detection (FIG. 8b). An overnight target hybridization was performed in the presence of 46% FM, 4×SSC/0.04% TW20, 0.01% SDS in a 5 ul reaction. Slide was washed 3 times with 0.5N NaNO₃/0.02% TW20/0.01% SDS and twice with 0.2×SSC followed by probe hybridization (32% FM, 4×SSC/0.04% TW20/0.005% SDS) for 25 min. The slide was again washed 3 times with 0.5N NaNO₃/0.02% TW20/0.005% SDS and twice with 0.5N NaNO₃ followed by silver development and light scattering detection.

These results indicated that the sensitivity of nanoparticle detection was 25-100 folds higher than fluorescent detection in the human RNA detection assay.

To examine the sensitivity of RNA detection and establish a correlation between signal intensity and mRNA copy numbers, a titration of bacterial control RNA was performed with improved assay conditions. The control RNA spike 4 (1,000 bases, in vitro transcript, Ambion cat# 1780 ArrayControl™ RNA Spikes) was included in human total RNA mix with dilutions from 0.5 pg/hybridization to 0.5 fg/hybridization. The hybridization results showed lower limitation of detection at 5-50 fg of mRNA (equivalent to 10,000-100,000 copies) with overnight hybridization and 50-500 fg (equivalent to 100,000-1000,000 copies) with 2 hour hybridization (FIG. 9).

1. A method for detecting or quantitating gene expression in a sample, said sample believed to have one or more different types of unlabeled target nucleic acids, each type of target nucleic acid having an oligonucleotide tail, said method comprising:
providing a substrate having a plurality of types of capture nucleic acid sequences attached thereto in an array for the detection of multiple portions of a target nucleic acid, the detection of multiple different target nucleic acids, or both;
providing nanoparticles having oligonucleotides bound thereto, the oligonucleotides bound to the nanoparticles having a sequence that is complementary to at least a portion of the oligonucleotide tail;
contacting the sample, the substrate, and the nanoparticles, said contracting occurring under conditions effective for hybridization of the target nucleic acids to the capture nucleic acid sequences bound to the substrate and hybridization of the target nucleic acids to the nanoparticles; and
observing a detectable change.

2. The method of claim 1 wherein the target nucleic acid is RNA or DNA.

3. The method of claim 2 wherein the target nucleic acid is mRNA.

4. The method of claim 2 wherein the target nucleic acid is cDNA.

5. The method of claim 1 wherein the oligonucleotide tail comprises a poly dT, a poly dA, or a synthetic oligonucleotide having a predetermined sequence.

6. The method of claim 1 wherein the oligonucleotides bound to the nanoparticles comprise a poly dT, a poly dA, or a synthetic oligonucleotide having a predetermined sequence.

7. The method of claim 1 wherein the capture nucleic acid sequences comprise an oligonucleotide, cDNA, or genomic sequence fragment.

8. The method of claim 1 wherein the sample is first contacted with the substrate, said contacting occurring under conditions effective for hybridization of the target nucleic acids with the capture nucleic acid sequence bound to the substrate, and then contacting the target nucleic acid bound to the substrate with the nanoparticles, said contacting occurring under conditions effective for hybridization of the target nucleic acids bound to the substrate with the oligonucleotides bound to the nanoparticles.

9. The method of claim 1 wherein the sample is first contacted with the nanoparticles, said contacting occurring under conditions effective for hybridization of the target nucleic acids with the oligonucleotides bound to the nanoparticles, and then contacting the target nucleic acid bound to the nanoparticles with the substrate, said contacting occurring under conditions effective for hybridization of the target nucleic acids bound to the nanoparticles with the capture nucleic acid sequences bound to the substrate.

10. The method of claim 1 wherein the sample, nanoparticles and substrate are contacted simultaneously under conditions effective for hybridization of the target nucleic acids with the oligonucleotides bound to the nanoparticles and with the capture nucleic acid sequences bound to the substrate.

11. The method of claim 1 wherein the nanoparticles are made of gold.

12. The method of claim 1 wherein the detectable change is observed after contacting the substrate having target nucleic acids and nanoparticles with a staining material.

13. The method of claim 12 wherein the staining material is silver stain.

14. A method for detecting or quantitating gene expression in a sample, said sample believed to have one or more different types of unlabeled target ribonucleic acids, each type of target ribonucleic acid including a poly dA olignonucleotide tail or a synthetic oligonucleotide tail of a predetermined sequence, said method comprising:
providing a substrate having a plurality of types of capture nucleic acid sequences attached thereto in an array for the detection of multiple portions of a target ribonucleic acid, the detection of multiple different target ribonucleic acids, or both;
providing nanoparticles having bound thereto poly dT oligonucleotides or a synthetic oligonucleotide sequence complementary to the sequence of the oligonucleotide tail;
contacting the sample, the substrate, and the nanoparticles, said contracting occurring under conditions effective for hybridization of the target ribonucleic acids to the capture nucleic acid sequences to the substrate and hybridization of the target ribonucleic acids to the nanoparticles; and
contacting the nanoparticles bound to the support with a staining material to produce a detectable change; and
observing the detectable change.

15. The method of claim 14 wherein the sample is first contacted with the substrate, said contacting occurring under conditions effective for hybridization of the target ribonucleic acids with the capture nucleic acid sequences bound to the substrate, and then contacting the target ribonucleic acid bound to the substrate with the nanoparticles, said contacting occurring under conditions effective for hybridization of the target ribonucleic acids bound to the substrate with the oligonucleotides bound to the nanoparticles.

16. The method of claim 14 wherein the sample is first contacted with the nanoparticles, said contacting occurring under conditions effective for hybridization of the target ribonucleic acids with the oligonucleotides bound to the nanoparticles, and then contacting the target ribonucleic acid bound to the nanoparticles with the substrate, said contacting occurring under conditions effective for hybridization of the target ribonucleic acids bound to the nanoparticles with the capture nucleic acid sequences bound to the substrate.

17. The method of claim 14 wherein the sample, nanoparticles and substrate are contacted simultaneously under conditions effective for hybridization of the target nucleic acids with the oligonucleotides bound to the nanoparticles and with the capture nucleic acid sequences bound to the substrate.

18. The method of claim 14 wherein the nanoparticles are made of gold.

19. The method of claim 14 wherein the staining material is silver stain.

20. The method of claim 14 wherein the capture nucleic acid sequences comprise an oligonucleotide, cDNA, or genomic sequence fragment.

21. A method for detecting or quantitating gene expression in a sample, said sample believed to have one or more different types of target cDNAs, each type of target cDNA including a poly dT olignonucleotide tail or a synthetic oligonucleotide tail having a predetermined sequence, said method comprising:
providing a substrate having a plurality of types of capture nucleic acid sequences attached thereto in an array for the detection of multiple portions of a target ribonucleic acid, the detection of multiple different target ribonucleic acids, or both;
providing nanoparticles having bound thereto poly dA oligonucleotides or synthetic oligonucleotides having a predetermined sequence;
contacting the sample, the substrate, and the nanoparticles, said contracting occurring under conditions effective for hybridization of the target cDNAs to the capture nucleic acid sequences bound to the substrate and hybridization of the target cDNAs to the nanoparticles; and
contacting the nanoparticles bound to the support with a staining material to produce a detectable change; and
observing the detectable change.

22. The method of claim 21 wherein the sample is first contacted with the substrate, said contacting occurring under conditions effective for hybridization of the target cDNAs with the capture nucleic acid sequences bound to the substrate, and then contacting the target cDNAs bound to the substrate with the nanoparticles, said contacting occurring under conditions effective for hybridization of the target cDNAs bound to the substrate with the oligonucleotides bound to the nanoparticles.

23. The method of claim 21 wherein the sample is first contacted with the nanoparticles, said contacting occurring under conditions effective for hybridization of the target cDNAs with the oligonucleotides bound to the nanoparticles, and then contacting the target cDNAs bound to the nanoparticles with the substrate, said contacting occurring under conditions effective for hybridization of the target cDNAs bound to the nanoparticles with the capture nucleic acid sequences bound to the substrate.

24. The method of claim 21 wherein the target cDNAs, nanoparticles and substrate are contacted simultaneously under conditions effective for hybridization of the target cDNAs with the oligonucleotides bound to the nanoparticles and with the capture nucleic acid sequences bound to the substrate.

25. The method of claim 21 wherein the nanoparticles are made of gold.

26. The method of claim 21 wherein the staining material is silver stain.

27. The method of claim 21 wherein the capture nucleic acid sequences comprise an oligonucleotide, cDNA, or genomic sequence fragment.

28. A kit for detecting or quantitating gene expression in a sample, said sample believed to have one or more different types of unlabeled target nucleic acids, each type of target nucleic acid including a poly dT, poly dA oligonucleotide tail, or a synthetic oligonucleotide tail having a predetermined sequence, said kit comprising:
a substrate having a plurality of types of capture nucleic acid sequences attached thereto in an array for the detection of multiple portions of a target nucleic acid, the detection of multiple different target nucleic acids, or both; and
one or more types of nanoparticles having bound thereto poly dT oligonucleotides, poly dA oligonucleotides, or synthetic oligonucleotides having a predetermined sequence.

29. The method of claim 28 wherein the nanoparticles are made of gold.

30. The method of claim 28 wherein the capture nucleic acid sequences comprise an oligonucleotide, cDNA, or genomic sequence fragment.


SEQUENCE LISTING (24 sequences)

SEQ ID NO: 1 - DNA, 70 bp, Artificial
  cytochrome c oxidase subunit Vic sense oligo
ctgtttgtcactgggtgacctcccgtccttgtgggcgctccacgggccctggtctacgggccttcatgag

SEQ ID NO: 2 - DNA, 70 bp, Artificial
  Beta actin sense oligo
tggaacggtgaaggtgacagcagtcggttggagcgagcatcccccaaagttcacaatgtggccgaggact

SEQ ID NO: 3 - DNA, 50 bp, Artificial
  glutamyl-prolyl-tRNA synthetase capture sequence 1
gagggtttccaggtttatattcctggccagttttctccttatattcagct

SEQ ID NO: 4 - DNA, 50 bp, Artificial
  Homo sapiens cDNA clone IMAGE4093756, partial cds capture sequence 2
acacatccgtctcctctgcgatataaccaaatggtgtttgacggttgaat

SEQ ID NO: 5 - DNA, 50 bp, Artificial
  Homo sapiens cDNA clone IMAGE4093756, partial cds capture sequence 2B
ttaatgtttctaacaaagcgtatcatgcaaacggagattagaggttatac

SEQ ID NO: 6 - DNA, 50 bp, Artificial
  hypothetical protein FLJ14668 capture sequence 3
taagggagtcagctcatcctagcccaagttgcttactttttctcccttga

SEQ ID NO: 7 - DNA, 50 bp, Artificial
  3-ketoacyl CoA thiolase beta-subunit of mitochondrial trifunctional protein, exon 8, 9, 10 capture sequence 4
ccgtagggcttgatgaatgcaggttttagtttggccatctgctccagtga

SEQ ID NO: 8 - DNA, 50 bp, Artificial
  chromatin assembly factor 1, subunit B (p60) capture sequence 5
tgtgtgcactttcacgaggatgccagggaggactcactgattttcacact

SEQ ID NO: 9 - DNA, 50 bp, Artificial
  chromatin assembly factor 1, subunit B (p60) capture sequence 5B
atactctaaaattcgacagagtaaaatctcaaattactttctcatcttcc

SEQ ID NO: 10 - DNA, 50 bp, Artificial
  transcription factor 3, TCF3 capture sequence 6
actgctgtttcttcctcctcgcgctgggtgaatctcgtttgaattctatg

SEQ ID NO: 11 - DNA, 50 bp, Artificial
  cDNA FLJ37123 fis capture sequence 7
cggaagttggaggcgtcatgcagcgcctcctgcctgggagccaggcgatc

SEQ ID NO: 12 - DNA, 49 bp, Artificial
  cDNA FLJ37123 fis capture sequence 7S
atcgcctggctcccaggcaggaggcgctgcatgacgcctccaacttccg

SEQ ID NO: 13 - DNA, 50 bp, Artificial
  adenosine monophosphate deaminase 2, isoform L capture sequence 8
aacaccactcccggggttgagtggcagatccaggactttgcagcaactgt

SEQ ID NO: 14 - DNA, 50 bp, Artificial
  adenosine monophosphate deaminase 2, isoform L capture sequence 8B
tatgaaacactgcagttcacagcaaaggcctcagtccagaacacaacata

SEQ ID NO: 15 - DNA, 50 bp, Artificial; identical to SEQ ID NO: 8
  chromatin assembly factor 1, subunit B (p60) capture sequence 9

SEQ ID NO: 16 - DNA, 50 bp, Artificial
  isoleucine-tRNA synthetase capture sequence 10
tgtaacctgctcccaacatgactgcataggtgtctaaggttaagtgtgaa

SEQ ID NO: 17 - DNA, 50 bp, Artificial
  seryl-tRNA synthetase capture sequence 11
tggtttcatcagtcatcaatgatgggtccctatgcccatgcgaggagaca

SEQ ID NO: 18 - DNA, 50 bp, Artificial
  Ribosomal Protein L32 capture sequence 12
tactcattttcttcactgcgcagcctggcattggggttggtgactctgat

SEQ ID NO: 19 - DNA, 50 bp, Artificial
  actin, beta capture sequence 13
actgggccattctccttagagagaagtggggtggcttttaggatggcaag

SEQ ID NO: 20 - DNA, 49 bp, Artificial
  actin, beta capture sequence 13S
ttgccatcctaaaagccaccccacttctctctaaggagaatggcccagt

SEQ ID NO: 21 - DNA, 50 bp, Artificial
  ubiquitin B capture sequence 14
atcttggccttcacattttcgatggtgtcactgggctccacctccagagt

SEQ ID NO: 22 - DNA, 20 bp, Artificial
  detection probe
aaaaaaaaaaaaaaaaaaaa

SEQ ID NO: 23 - DNA, 20 bp, Artificial
  detection probe
tttttttttttttttttttt

SEQ ID NO: 24 - DNA, 20 bp, Artificial
  detection probe
aaaaaaaaaaaaaaaaaaaa